(19) United States
(12) Patent Application Publication
(10) Pub. No.: US 2015/0274870 A1
(43) Pub. Date: Oct. 1, 2015

(54) PERFLUOROALKYLENE ETHER-CONTAINING COMPOUND AND SURFACE PROTECTIVE FILM

(71) Applicant: FUJI XEROX CO., LTD., Tokyo (JP)

(72) Inventors: Hiroshi SAEGUSA, Kanagawa (JP); Tatsuki SANO, Kanagawa (JP); Tomoko MIYAHARA, Kanagawa (JP); Kaoru TORIKOSHI, Kanagawa (JP)

(73) Assignee: FUJI XEROX CO., LTD., Tokyo (JP)

(21) Appl. No.: 14/608,404

(22) Filed: Jan. 29, 2015

(30) Foreign Application Priority Data
Mar. 26, 2014 (JP) 2014-064297

(51) Int. Cl. C08F 222/20; C09D 135/02; C07C 69/734

(52) U.S. Cl. CPC C08F 222/20; C07C 69/734; C09D 135/02

(57) ABSTRACT

Provided is a perfluoroalkylene ether-containing compound represented by the following general formula (1):

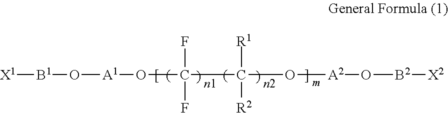

wherein R¹ and R² each independently indicates a fluorine atom or a trifluoromethyl group, provided that both R¹ and R² are not the fluorine atom, n1 indicates an integer from 1 to 5, n2 indicates an integer from 0 to 2, the total number of n1 and n2 is less than or equal to 5, m indicates an integer greater than or equal to 1, A¹ and A² each independently indicates a bivalent group represented by the following general formula (2), B¹ and B² each independently indicates a bivalent group selected from the group consisting of a single bond and the following (B-1) to (B-3), X¹ and X² each independently indicates a monovalent group having at least one reactive crosslinking group selected from the group consisting of the following (X-1) to (X-8).

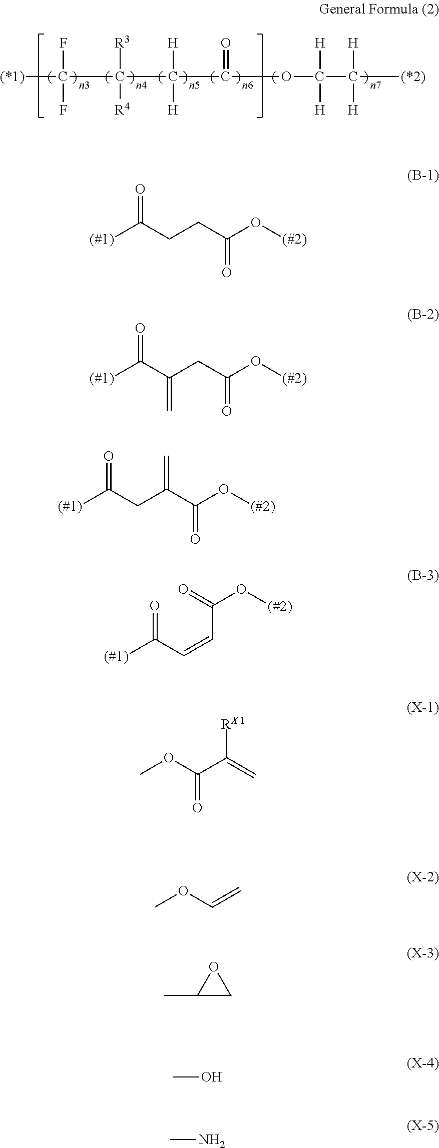

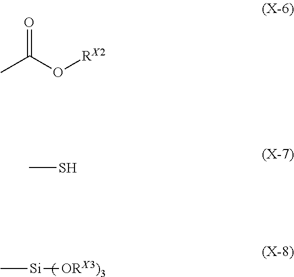

### CROSS-REFERENCE TO RELATED APPLICATIONS

This application is based on and claims priority under 35 USC 119 from Japanese Patent Application No. 2014-064297 filed Mar. 26, 2014.

### BACKGROUND

1. Technical Field

The present invention relates to a perfluoroalkylene ether-containing compound and a surface protective film.

2. Related Art

In the related art, a surface protective film is disposed on a surface from a viewpoint of preventing a scratch on the surface in various fields.

### SUMMARY

According to an aspect of the invention, there is provided a perfluoroalkylene ether-containing compound represented by the following general formula (1):

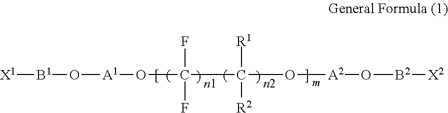

wherein in the general formula (1), R¹ and R² each independently indicates a fluorine atom or a trifluoromethyl group, provided that both R¹ and R² are not the fluorine atom;

n1 indicates an integer from 1 to 5, n2 indicates an integer from 0 to 2, and the total number of n1 and n2 is less than or equal to 5; m indicates an integer greater than or equal to 1;

A¹ and A² each independently indicates a bivalent group represented by the following general formula (2);

B¹ and B² each independently indicates a bivalent group selected from the group consisting of a single bond and the following (B-1) to (B-3);

X¹ and X² each independently indicates a monovalent group having at least one reactive crosslinking group selected from the group consisting of the following (X-1) to (X-8); X¹ and X² may each independently have one or more groups having a structure obtained by excluding X¹ or X² from the general formula (1); provided that when B¹ is a single bond or the following (B-1), X¹ indicates a monovalent group having two or more reactive crosslinking groups or one or more reactive crosslinking groups and one or more groups having a structure obtained by excluding X¹ from the general formula (1), and when B² is a single bond or the following (B-1), X² indicates a monovalent group having two or more reactive crosslinking group or one or more reactive crosslinking groups and one or more groups having a structure obtained by excluding X² from the general formula (1),

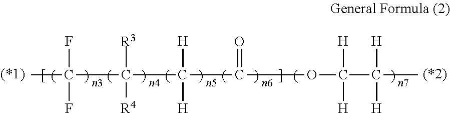

wherein in the general formula (2), R³ and R⁴ each independently indicates a fluorine atom or a trifluoromethyl group, provided that both R³ and R⁴ are not the fluorine atom;

n3 indicates an integer from 0 to 5, n4 indicates an integer from 0 to 2, n5 indicates an integer greater than or equal to 0, n6 indicates 0 or 1, and n7 indicates an integer greater than or equal to 0, provided that all of n3, n4, n5, and n6 are not 0;

a bivalent group represented by the general formula (2) is bonded to a perfluoroalkylene ether structure in a (*1) portion;

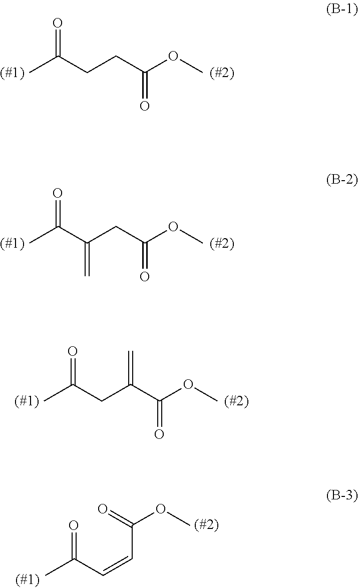

(B-1) to (B-3) are respectively bonded to X¹ or X² in a (#2) portion;

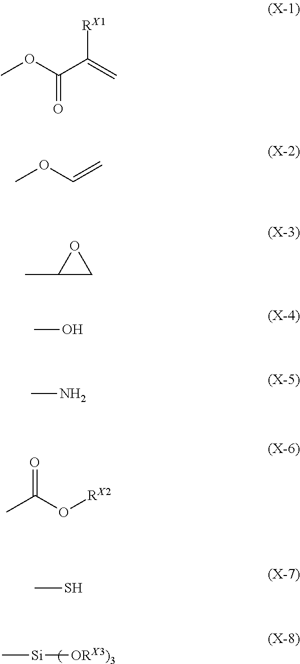

R^{X1} in (X-1) indicates a hydrogen atom, a methyl group, or a trifluoromethyl group; R^{X2} in (X-6) indicates a hydrogen atom or an alkyl group; and R^{X3} in (X-8) indicates an alkyl group.

### BRIEF DESCRIPTION OF THE DRAWINGS

Exemplary embodiments of the present invention will be described in detail based on the following figures, wherein:

FIG. 1 is a perspective view illustrating a schematic configuration of an endless belt according to this exemplary embodiment;

FIG. 2 is a cross-sectional view of the endless belt according to this exemplary embodiment;

FIG. 3 is a schematic configuration diagram illustrating an image forming apparatus using the endless belt according to this exemplary embodiment;

FIG. 4 is a schematic configuration diagram illustrating the image fixing device using the endless belt according to this exemplary embodiment;

FIG. 5 is a graph illustrating identification data (an IR chart) of a perfluoroalkylene ether-containing compound 4 obtained in Example;

FIG. 6 is a graph illustrating identification data (a 1H-NMR chart) of the perfluoroalkylene ether-containing compound 4 obtained in Example;

FIG. 7 is a graph illustrating identification data (an IR chart) of the perfluoroalkylene ether-containing compound 5 obtained in Example;

FIG. 8 is a graph illustrating an IR chart of a surface protective film sample (before heating) obtained in Example 4; and

FIG. 9 is a graph illustrating an IR chart of a surface protective film sample (before heating) obtained in Comparative Example 1.

### DETAILED DESCRIPTION

Hereinafter, exemplary embodiments of the invention will be described in detail.

A perfluoroalkylene ether-containing compound according to this exemplary embodiment is represented by the following general formula (1).

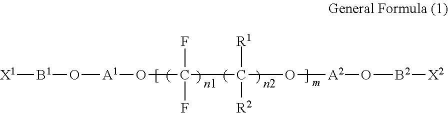

In the general formula (1), R¹ and R² each independently indicates a fluorine atom or a trifluoromethyl group, provided that both R¹ and R² are not the fluorine atom.

n1 indicates an integer from 1 to 5, n2 indicates an integer from 0 to 2, and the total number of n1 and n2 is less than or equal to 5. m indicates an integer greater than or equal to 1.

A¹ and A² each independently indicates a bivalent group represented by the following general formula (2).

B¹ and B² each independently indicates a bivalent group selected from the group consisting of a single bond and the following (B-1) to (B-3).

X¹ and X² each independently indicates a monovalent group having at least one reactive crosslinking group selected from the group consisting of the following (X-1) to (X-8). Furthermore, X¹ and X² may each independently has one or more groups having a structure obtained by excluding X¹ or X² from the general formula (1). When B¹ is a single bond or the following (B-1), X¹ indicates a monovalent group having two or more reactive crosslinking groups or one or more reactive crosslinking groups, and one or more groups having a structure obtained by excluding X¹ from general formula (1). In addition, when B² is a single bond or the following (B-1), X² indicates a monovalent group having two or more reactive crosslinking groups or one or more reactive crosslinking groups, and one or more groups having a structure obtained by excluding X² from general formula (1).

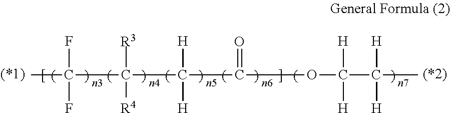

In the general formula (2), R³ and R⁴ each independently indicates a fluorine atom or a trifluoromethyl group, provided that both R³ and R⁴ are not the fluorine atom.

n3 indicates an integer from 0 to 5, n4 indicates an integer from 0 to 2, n5 indicates an integer greater than or equal to 0, n6 indicates 0 or 1, and n7 indicates an integer greater than or equal to 0, provided that all of n3, n4, n5, and n6 are not 0.

Furthermore, a bivalent group represented by the general formula (2) is bonded to a perfluoroalkylene ether structure in a (*1) portion.

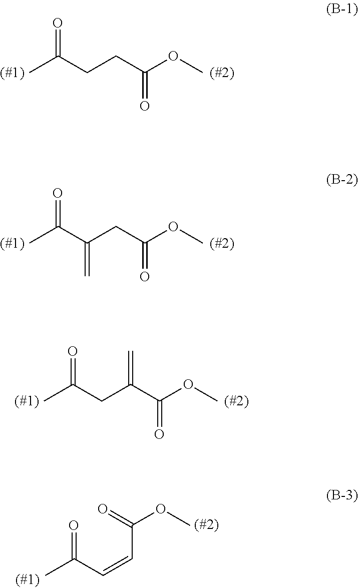

(B-1) to (B-3) are respectively bonded to X¹ or X² in a (#2) portion.

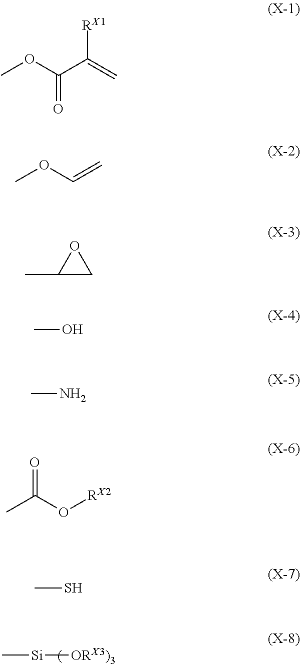

R^{X1} in (X-1) indicates a hydrogen atom, a methyl group, or a trifluoromethyl group. R^{X2} in (X-6) indicates a hydrogen atom or an alkyl group. R^{X3} in (X-8) indicates an alkyl group.

Recently, a surface protective film has been disposed from a viewpoint of preventing a scratch on a surface in various fields. In the surface protective film, there is a demand for a releasing property in addition to damage resistance from a viewpoint of an antifouling property of a surface, and for example, a crosslinked fluorine resin material is used. Specifically, there is an attempt to use a urethane (meth)acrylate compound in which an isocyanate group in a trivalent or higher valent polyisocyanate compound, a hydroxyl group of a fluorine-containing alcohol compound, and a hydroxyl group of a hydroxyl group-containing (meth)acrylate compound respectively form a urethane bond.

However, in the fluorine resin material, heat resistance is not able to be obtained due to the urethane bond, and thus the fluorine resin material is not able to be used in a high temperature environment.

In contrast, a perfluoroalkylene ether-containing compound according to this exemplary embodiment has a structure represented by the general formula (1), thus heat resistance is obtained, and in a crosslinked product obtained by performing crosslinking polymerization of the perfluoroalkylene ether-containing compound, excellent damage resistance is also able to be obtained.

It is considered that B¹ and B² bonding a perfluoroalkylene ether structure portion (a portion surrounded by [ ]ₘ) to a portion (X¹ and X²) having a reactive crosslinking group on a terminal are represented by a bivalent group selected from the group consisting of a single bond and (B-1) to (B-3), and have any one structure of the single bond and (B-1) to (B-3) without including a urethane bond in B¹ and B², and thus in this exemplary embodiment, excellent heat resistance is obtained.

In addition, in the compound according to this exemplary embodiment, when B¹ (or B²) is a bivalent group represented by (B-2) or (B-3), X¹ (or X²) is a monovalent group having at least one reactive crosslinking group selected from the group consisting of (X-1) to (X-8). That is, B¹ (or B²) has a carbon-carbon double bond which is also a reactive crosslinking group, X¹ (or X²) has one or more reactive crosslinking groups, and one terminal has at least two crosslinking groups. Accordingly, by performing crosslinking polymerization of the compound described above, a crosslinked product in which a group having a structure obtained by excluding X¹ (or X²) from the general formula (1) is crosslinked polymerized into at least three sections starting from —B¹—X¹ (or —B²—X²) is obtained.

Further, in the compound according to this exemplary embodiment, when B¹ (or B²) is a bivalent group represented by a single bond or (B-1), X¹ (or X²) has two or more reactive crosslinking groups, or has one or more reactive crosslinking groups, and has one or more groups having a structure obtained by excluding X¹ from the general formula (1). Accordingly, by performing crosslinking polymerization of the compound, a crosslinked product in which a group having a structure obtained by excluding X¹ (or X²) from the general formula (1) is crosslinked polymerized into at least three sections starting from X¹ (or X²) is obtained.

It is considered that perfluoroalkylene ether having an excellent releasing property also has excellent flexibility, and a terminal of a main chain having a perfluoroalkylene ether structure which is excellent in flexibility is fixed to form crosslinking polymerization of at least three sections as described above, and thus in this exemplary embodiment, excellent damage resistance is expressed.

Perfluoroalkylene Ether-Containing Compound

A structure of the perfluoroalkylene ether-containing compound according to this exemplary embodiment which is represented by the general formula (1) will be described in more detail.

First, the general formula (1) includes the perfluoroalkylene ether structure portion surrounded by [ ]ₘ. In the perfluoroalkylene ether structure portion, R¹ and R² each independently indicates a fluorine atom or a trifluoromethyl group, provided that both R¹ and R² are not the fluorine atom.

n1 indicates an integer from 1 to 5, n2 indicates an integer from 0 to 2, and the total number of n1 and n2 is less than or equal to 5. Further, n1 is preferably from 1 to 3, n2 is preferably 0 or 1, and the total number of n1 and n2 is preferably from 1 to 3.

m which is the number of [ ] surrounding the perfluoroalkylene ether structure portion indicates an integer greater than or equal to 1. Furthermore, plural perfluoroalkylene ether structures (—(CF₂)ₙ₁—(C(R¹) (R²))ₙ₂—O—) when m is greater than or equal to 2 may have the same structure or may have a different structure. m is preferably from 2 to 100, and is more preferably from 5 to 50.

As a specific example of the perfluoroalkylene ether structure portion ([—(CF₂)ₙ₁—(C(R¹) (R²))ₙ₂—O—]ₘ), for example, structures of the following (m-1) to (m-8) are included. Furthermore, m1 and m2 represented in (m-2), (m-3), and (m-4) each independently indicates an integer greater than or equal to 1, and the total number of m1 and m2 is m.

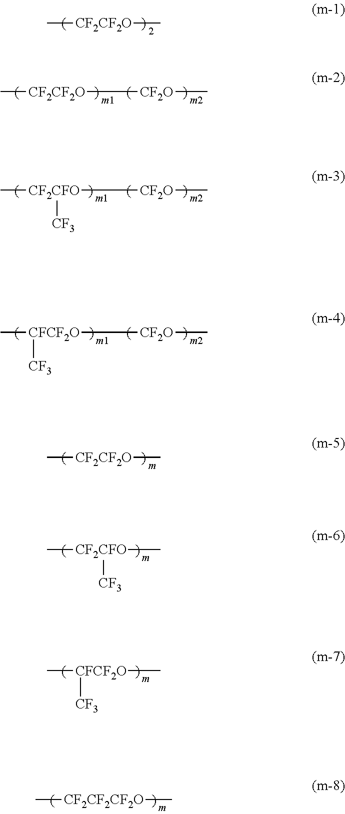

Furthermore, among the structures of (m-1) to (m-8), the structures of (m-2), (m-6), (m-7), and (m-8) are preferable, and the structure of (m-2) is more preferable.

In the general formula (1), A¹ and A² each independently indicates a bivalent group represented by the general formula (2).

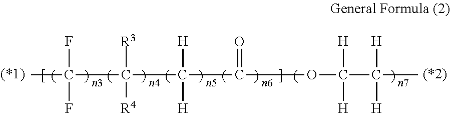

In the general formula (2), R³ and R⁴ each independently indicates a fluorine atom or a trifluoromethyl group, provided that both R³ and R⁴ are not the fluorine atom.

n3 indicates an integer from 0 to 5, n4 indicates an integer from 0 to 2, n5 indicates an integer greater than or equal to 0, n6 indicates 0 or 1, and n7 indicates an integer greater than or equal to 0, provided that all of n3, n4, n5, and n6 are not 0.

Furthermore, the bivalent group represented by the general formula (2) is bonded to the perfluoroalkylene ether structure in a (*1) portion, and is bonded to (—O—B¹—X¹) or (—O—B²—X²) in a (*2) portion.

In addition, in the general formula (2), the total number of n5 and n6 is preferably less than or equal to 2, and the total number of n5 and n6 is more preferably less than or equal to 1.

A preferable structure of the bivalent group represented by the general formula (2) is as follows.

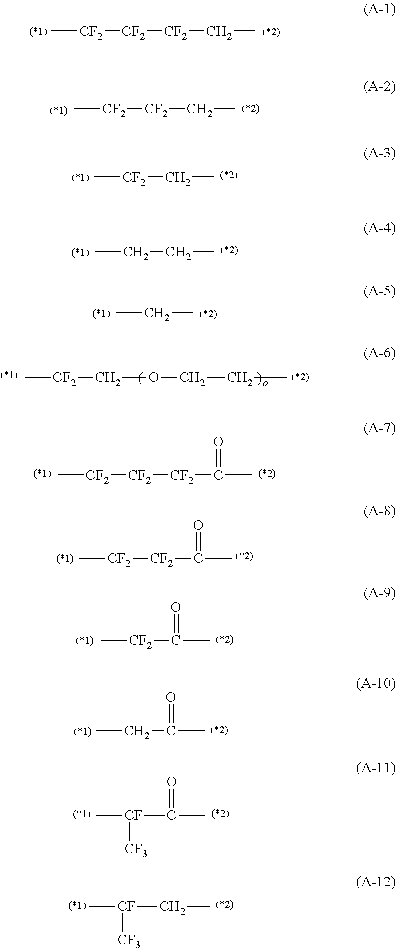

o in (A-6) indicates an integer greater than or equal to 1, preferably from 1 to 50, and more preferably from 1 to 20.

Furthermore, among structures of (A-1) to (A-12), in particular, the structures of (A-1), (A-2), (A-3), (A-6), (A-8), (A-11), and (A-12) are more preferable.

In the general formula (1), B¹ and B² each independently indicates a bivalent group selected from the group consisting of a single bond and the following (B-1) to (B-3).

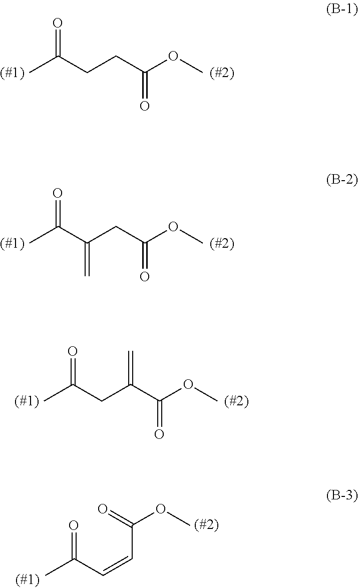

(B-1) to (B-3) are respectively bonded to X¹ or X² in a (#2) portion, and to (—O—A¹ . . . ) or (—O—A² . . . ) side in a (#1) portion.

Furthermore, among the structures, in particular, the structure of (B-1) is more preferable as B¹ and B².

In the general formula (1), when B¹ (or B²) is (B-2) or (B-3), that is, B¹ (or B²) has a reactive crosslinking group, X¹ (or X²) indicates a monovalent group having at least one reactive crosslinking group selected from the group consisting of the following (X-1) to (X-8).

In addition, when B¹ (or B²) is a single bond or (B-1), that is, B¹ (or B²) does not have a reactive crosslinking group, X¹ (or X²) indicates a monovalent group having two or more reactive crosslinking groups described below, or one or more reactive crosslinking groups and one or more groups having a structure obtained by excluding X¹ (or X²) from the general formula (1).

In crosslinked product which is obtained by performing crosslinking polymerization of the compound, when X¹ and X², and B¹ and B² satisfy the configuration, a structure in which the group having the structure obtained by excluding X¹ (or X²) from the general formula (1) is crosslinked polymerized into at least three sections starting from —B¹—X¹ and —B²—X² is obtained.

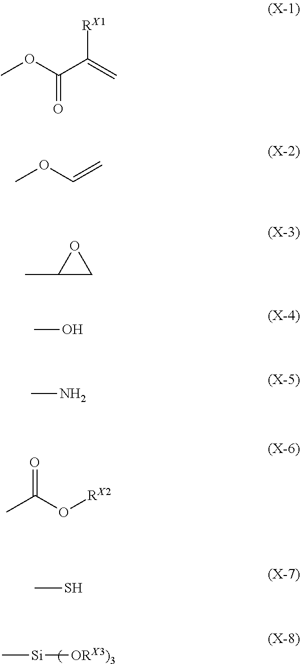

R^{X1} in (X-1) indicates a hydrogen atom (that is, a crosslinking group=an alkyl group), a methyl group (that is, a crosslinking group=a methacryl group), or a trifluoromethyl group.

Furthermore, among them, as R^{X1}, the hydrogen atom and the trifluoromethyl group are more preferable.

A crosslinking group represented by (X-3) indicates an epoxy group, a crosslinking group represented by (X-4) indicates a hydroxyl group, and a crosslinking group represented by (X-5) indicates an amino group.

R^{X2} in (X-6) indicates a hydrogen atom (that is, a crosslinking group=a carboxy group), or an alkyl group (that is, a crosslinking group=an ester group).

Furthermore, as the alkyl group represented by R^{X2} in (X-6), an alkyl group having carbon atoms from 1 to 18 is preferable, and an alkyl group having carbon atoms from 1 to 4 is more preferable.

The alkyl group represented by R^{X2} may be any shape of a straight chain, a branched chain, and a cyclic, and as a specific example thereof, methyl, ethyl, n-propyl, isopropyl, n-butyl, isobutyl, n-pentyl, cyclopentyl, n-hexyl, cyclohexyl, 2-ethylhexyl, lauryl, stearyl, and the like are included.

Among them, as R^{X2}, methyl, ethyl, n-propyl, isopropyl, n-butyl, and isobutyl are more preferable.

The crosslinking group represented by (X-7) indicates a thiol group.

R^{X3} in (X-8) indicates an alkyl group (that is, a crosslinking group=a trialkoxysilyl group).

Furthermore, as the alkyl group represented by R^{X3} in (X-8), an alkyl group having carbon atoms from 1 to 10 is preferable, and an alkyl group having carbon atoms from 1 to 4 is more preferable.

The alkyl group represented by R^{X3} may be any shape of a straight chain, a branched chain, and a cyclic, and as a specific example thereof, methyl, ethyl, n-propyl, isopropyl, n-butyl, isobutyl, n-pentyl, cyclopentyl, n-hexyl, cyclohexyl, 2-ethylhexyl, lauryl, stearyl, and the like are included.

Among them, as R^{X3}, methyl, ethyl, n-propyl, isopropyl, n-butyl, and isobutyl are more preferable.

As the reactive crosslinking group included in X¹ and X², among structures of (X-1) to (X-8), in particular, the structures of (X-1), (X-2), (X-3), (X-5), (X-6), and (X-8) are preferable, and the structures of (X-1), (X-2), (X-3), and (X-8) are more preferable.

The number of reactive crosslinking groups included in X¹ and X² is preferably from 1 to 20, and is more preferably from 2 to 10.

X¹ and X² may have other linking groups between one or more reactive crosslinking groups and portions in which X¹ and X² are bonded to B¹ (or B²). That is, X¹ and X² may be configured of one or more reactive crosslinking groups, other bivalent or higher valent linking groups.

The linking group included in X¹ and X² is a bivalent or higher valent organic group, and for example, a bivalent or higher valent organic group formed of a structure in which one or two or more chains selected from an alkyl chain, an aromatic chain, an ether group (—O—), a carbonyl group (—CO—), and an ester group (—CO—O—) are combined.

As a specific example of the linking group, for example, the following organic groups (1) to (6) are included. Furthermore, in the following specific example of the linking group, X¹ and X² are bonded to B¹ or B² in the * portion, and X¹ and X² are bonded to the reactive crosslinking group in the # portion.

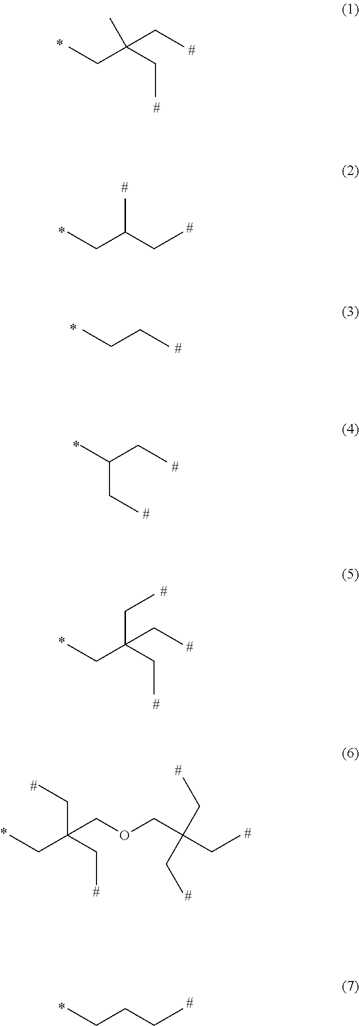

Furthermore, for example, the above linking group (3) has only one reactive crosslinking group, and thus in this exemplary embodiment, the linking group (3) is able to be used when X¹ (or X²) is (B-2) or (B-3).

In addition, as described above, X¹ and X² may have one or more groups having a structure obtained by excluding X¹ or X² from the general formula (1), and in this case, as a specific example of the linking group, for example, the following organic groups (7) to (12) are included. Furthermore, in the following specific example of the linking group, X¹ and X² are bonded to B¹ or B² in the * portion, and X¹ and X² are bonded to the reactive crosslinking group in the # portion.

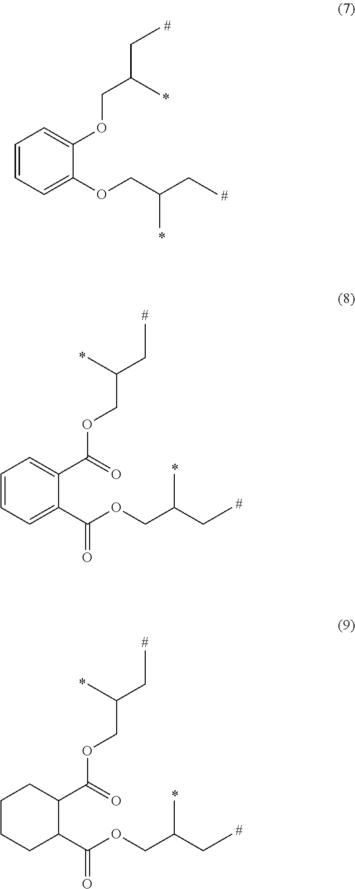 [SMILES: CCC(C)COc1ccccc1OCC(C)CC ; CCC(C)COC(=O)c1ccccc1C(=O)OCC(C)CC ; CCC(C)COC(=O)C1CCCCC1C(=O)OCC(C)CC]

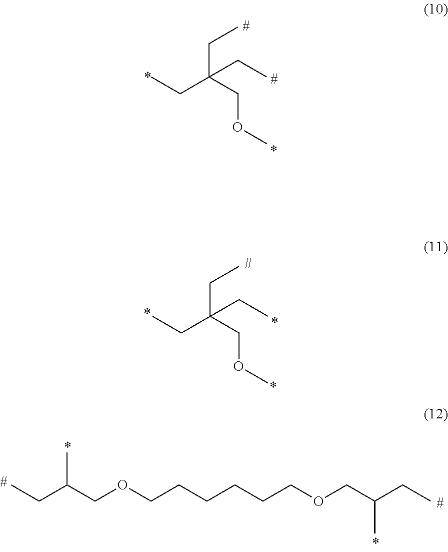

Here, a specific example of X¹ and X² is as follows.

First, as the specific example of X¹ and X², an aspect in which a group having a structure obtained by excluding X¹ or X² from the general formula (1) is not included is exemplified. As an example of an aspect having the crosslinking group of (X-1) as the reactive crosslinking group, the following (X-1a) to (X-1f) are included. Furthermore, R^{X1} of the following (X-1a) to (X-1f) has the same definition as that of R^{X1} of (X-1).

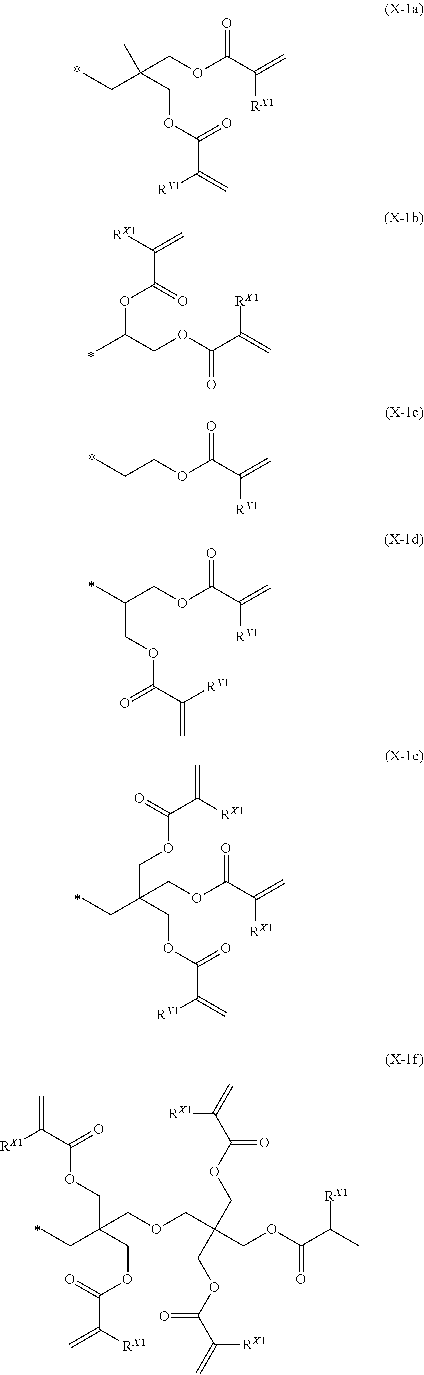

In addition, as an example of an aspect having the crosslinking group of (X-2) as the reactive crosslinking group, the following (X-2a) to (X-2f) are included.

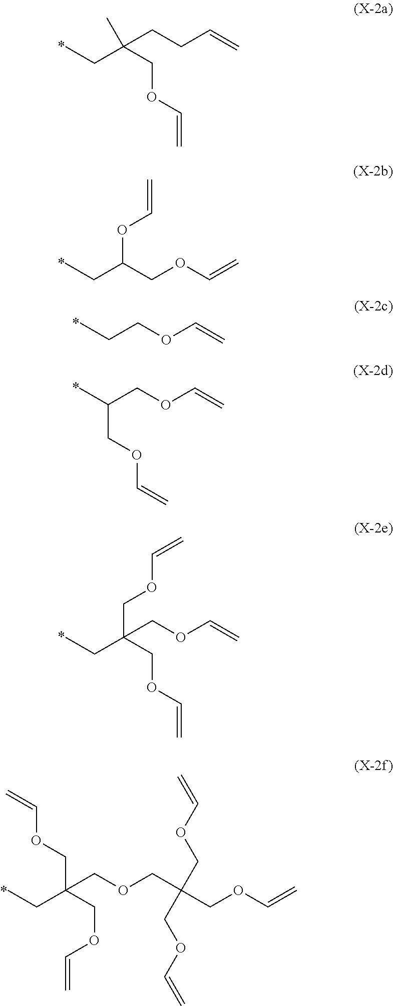

Similarly, as an example of an aspect having the crosslinking group of (X-3) to (X-8) as the reactive crosslinking group, a monovalent group in which the crosslinking group represented by (X-3) to (X-8) is bonded to the # portion of the linking group represented by (1) to (6) is included.

Next, as a specific example of X¹ and X², an aspect in which one or more groups having a structure obtained by excluding X¹ or X² from the general formula (1) is included is exemplified. As an example of an aspect having the crosslinking group of (X-1) as the reactive crosslinking group, the following (X-1g) to (X-11) are included. Furthermore, R^{X1} of the following (X-1g) to (X-11) has the same definition as that of R^{X1} of (X-1).

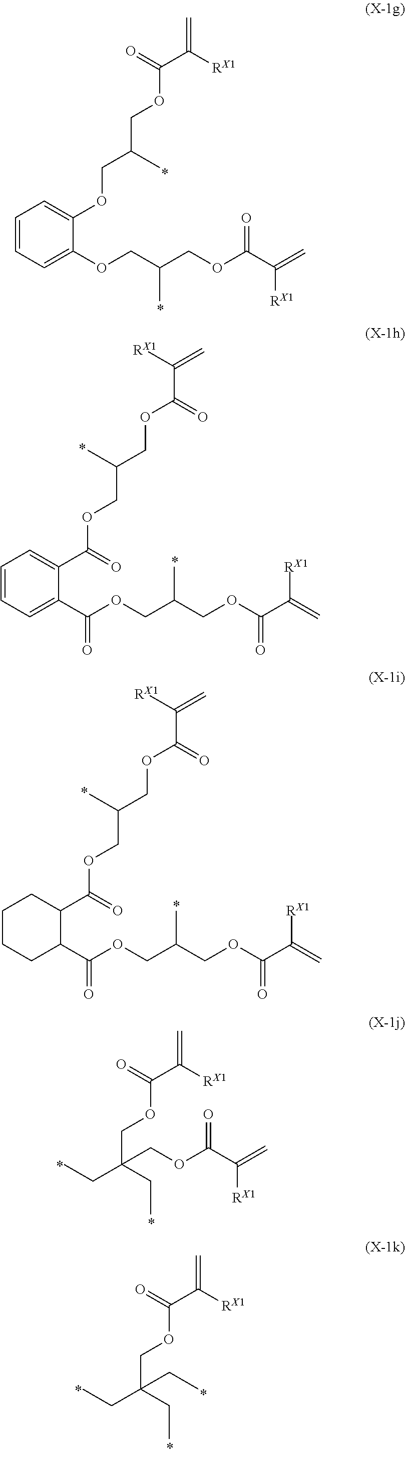

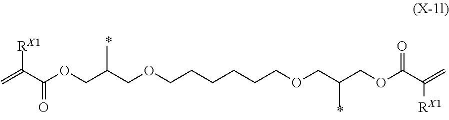

Similarly, as an example of an aspect having the crosslinking group of (X-2) to (X-8) as the reactive crosslinking group, a bivalent or higher valent group in which the crosslinking group of (X-2) to (X-8) is bonded to the # portion of the linking group represented by (7) to (12) is included.

Here, a specific example of the perfluoroalkylene ether-containing compound represented by the general formula (1) will be described. Here, the perfluoroalkylene ether-containing compound according to this exemplary embodiment is not limited to the following example.

| | | | | X¹, X² | |
|---|---|---|---|---|---|
| | | | | | Cross- |
| | Perfluoroalkylene | | | Linking | Linking |
| | Ether Structure | A¹, A² | B¹, B² | Group | Group |
| (1)-1 | (m-2) | (A-3) | Single Bond | (2) | (X-1) |
| (1)-2 | (m-2) | (A-3) | (B-3) | (3) | (X-1) |
| (1)-3 | (m-2) | (A-3) | (B-2) | (3) | (X-1) |
| (1)-4 | (m-2) | (A-3) | (B-1) | (4) | (X-1) |
| (1)-5 | (m-2) | (A-3) | (B-1) | (5) | (X-1) |
| (1)-6 | (m-2) | (A-3) | (B-1) | (6) | (X-1) |
| (1)-7 | (m-7) | (A-12) | (B-1) | (5) | (X-1) |
| (1)-8 | (m-7) | (A-11) | Single Bond | (5) | (X-1) |
| (1)-9 | (m-8) | (A-2) | (B-1) | (5) | (X-1) |
| (1)-10 | (m-8) | (A-8) | Single Bond | (5) | (X-1) |
| (1)-11 | (m-2) | (A-3) | Single Bond | (7) | (X-8) |
| (1)-12 | (m-2) | (A-3) | (B-1) | (5) | (X-4) |

Synthesis Method of Perfluoroalkylene Ether-Containing Compound

Next, an example of a synthesis method of the perfluoroalkylene ether-containing compound according to this exemplary embodiment which is represented by the general formula (1) will be described. Furthermore, a method of synthesizing the perfluoroalkylene ether-containing compound according to this exemplary embodiment is not limited to the following method.

For example, when a compound having a bivalent group represented by (B-1) as B¹ and B² of the general formula (1) is synthesized, as described in the following synthesis scheme, a compound (here, X* indicates X¹ or X² of the general formula (1)) having an OH group at a terminal of X* is reacted with succinic anhydride and an intermediate is synthesized, and the intermediate is reacted with a compound having an OH group on the outside of A¹ and A² of the general formula (1), and thus the perfluoroalkylene ether-containing compound represented by the general formula (1) is synthesized.

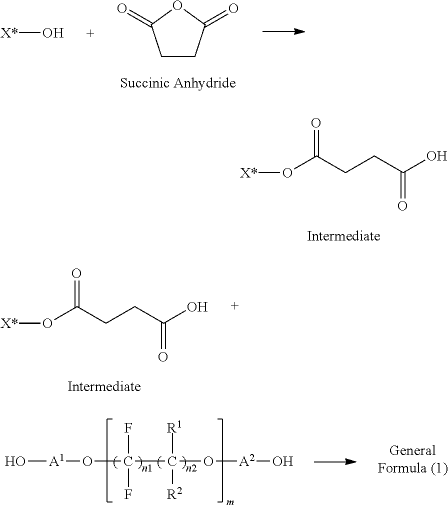

Furthermore, when a compound having a bivalent group represented by (B-2) and (B-3) as B¹ and B² is synthesized, the compound is synthesized by switching succinic anhydride of the synthesis scheme described above to itaconic acid anhydride or maleic acid anhydride.

In addition, when synthesizing a compound in which B¹ and B² have a single bond, the compound may be synthesized by directly reacting X*—OH and the compound having the OH group on the outside of A¹ and A² of the general formula (1) without performing a step of obtaining the intermediate by reacting a compound represented by X*—OH and succinic anhydride.

In addition, in a synthesis method of a compound of an aspect in which X¹ and X² has one or more groups having a structure obtained by excluding X¹ or X² from the general formula (1), for example, when it is a compound having the linking group represented by (10) or (11) as the “linking group” of X¹ and X², the compound is concurrently synthesized when a compound having the linking group represented by (5).

Surface Protective Film

Next, a surface protective film according to this exemplary embodiment will be described.

The surface protective film according to this exemplary embodiment has a structure in which the perfluoroalkylene ether-containing compound according to this exemplary embodiment described above is crosslinked polymerized.

Forming Method of Surface Protective Film (Crosslinking Polymerization Method)

The surface protective film according to this exemplary embodiment is formed by applying a coating liquid containing at least the perfluoroalkylene ether-containing compound (hereinafter, simply referred to as the “compound according to this exemplary embodiment”) according to this exemplary embodiment on a base material and by performing crosslinking polymerization of the compound.

Furthermore, in the surface protective film according to this exemplary embodiment, the compound according to this exemplary embodiment may be crosslinked polymerized through a crosslinking agent.

Here, for example, when a compound having the crosslinking group (an acryl group or the like) represented by (X-1) or (X-2) in the reactive crosslinking group of X¹ and X² is used as the compound according to this exemplary embodiment, it is possible to perform crosslinking polymerization without using a crosslinking agent.

In contrast, when a compound having the crosslinking group (that is, an epoxy group, a hydroxyl group, an amino group, a carboxyl group, or the like) represented by (X-3) to (X-8) in the reactive crosslinking group of X¹ and X² is used as the compound according to this exemplary embodiment, it is possible to perform the crosslinking polymerization by a method using the crosslinking agent as a curing agent, or a method using a combination (for example, a combination of a compound having an epoxy group in X¹ and X², and a compound having an amino group, a hydroxyl group, or a carboxyl group in X¹ and X²) in which the reactive crosslinking groups are reacted with each other.

Crosslinking Agent

As the crosslinking agent which is able to be used for the compound having the group (an epoxy group) represented by (X-3) as the reactive crosslinking group, pentaerythritol, dipentaerythritol, tripentaerythritol, polycarbonate diol, polyether diol, tris(2-hydroxyethyl)isocyanurate, and the like are included.

As the crosslinking agent which is able to be used for the compound having the group (a hydroxyl group, an amino group, a carboxyl group, or the like) represented by (X-4), (X-5), or (X-6) as the reactive crosslinking group, a crosslinking agent containing two or more epoxy groups is preferable. For example, 1,5-hexadiene diepoxide, 1,7-octadien diepoxide, neopentyl glycol diglycidyl ether, diglycidyl 1,2-cyclohexane dicarboxylate, 2,2-bis(4-glycidyl oxyphenyl)propane, triglycidyl isocyanurate, 1,6-bis(2,3-epoxypropoxy)naphthalene, and the like are included.

In addition, as the crosslinking agent which is able to be used for the compound having the group (an acryl group or the like) represented by (X-1) or (X-2) as the reactive crosslinking group, a crosslinking agent containing two or more acryl groups is preferable. For example, 2-hydroxy-3-acryloyloxypropyl methacrylate, polyethylene glycol diacrylate, tricyclodecanedimethanol diacrylate, 1,10-decanediol diacrylate, 1,6-hexanediol diacrylate, tripropylene glycol diacrylate, ethoxylated isocyanurate triacrylate, ε-caprolactone-modified tris-(2-acryloxyethyl)isocyanurate, pentaerythritol triacrylate, trimethylolpropane triacrylate, ditrimethylolpropane tetraacrylate, ethoxylated pentaerythritoltetraacrylate, pentaerythritoltetraacrylate, dipentaerythritol polyacrylate, dipentaerythritol hexaacrylate, and the like are included.

When the crosslinking agent is used, an amount thereof added to the compound according to this exemplary embodiment is preferably adjusted to be 1% to 500% with respect to weight of the compound according to this exemplary embodiment, and is more preferably adjusted to be 5% to 200%.

In addition, when a liquid compound is used as the compound according to this exemplary embodiment, the liquid compound may be used as the coating liquid.

When a compound which is able to be dissolved in a solvent, regardless of solid or liquid, is used as the compound according to this exemplary embodiment, the surface protective film is formed by dissolving the compound according to this exemplary embodiment, a curing agent (a crosslinking agent) when the curing agent (the crosslinking agent) is required, other additives, and the like in a solvent to prepare a coating liquid, applying the coating liquid on the base material, and performing the crosslinking polymerization.

In addition, when a solid compound which is not dissolved in the solvent is used as the compound according to this exemplary embodiment, the surface protective film is formed by heating the compound according to this exemplary embodiment, the curing agent (the crosslinking agent) when the curing agent (the crosslinking agent) is required, the other additives, and the like up to a temperature at which the compound according to this exemplary embodiment, the curing agent, the other additives, and the like are able to be dissolved, and performing the crosslinking polymerization.

Here, from a viewpoint of manufacturability, it is preferable that the surface protective film be formed by using a compound which is able to be dissolved in a solvent, or a compound which is a liquid at a normal temperature (25° C.)

As the solvent used for the coating liquid, for example, acetone, methyl ethyl ketone, methyl butyl ketone, methyl isobutyl ketone, cyclopentanone, cyclohexanone, ethyl acetate, propyl acetate, isopropyl acetate, butyl acetate, isobutyl acetate, amyl acetate, toluene, xylene, hexane, heptane, 1,4-dioxane, tetrahydrofuran, ethylene glycol monomethyl ether, ethylene glycol monoethyl ether, ethylene glycol monobutyl ether, ethylene glycol monoethyl ether acetate, methanol, ethanol, 1-propanol, 2-propanol, 1-butanol, tetrahydrofuran, 2H,3H-decafluoropentane, 1-methoxy-heptafluoropropane, 1-methoxy-nonafluorobutane, 1-ethoxy nonafluorobutane, and the like are included.

When the crosslinking polymerization is performed, energy may be supplied from the outside, and for example, the energy may be supplied by a unit emitting ultraviolet light, a unit emitting electron beams, and a heating unit.

In addition, a polymerization initiator for performing the crosslinking polymerization may be added. As a specific example of the polymerization initiator, for example, IRGACURE184, IRGACURE651, IRGACURE123, IRGACURE819, DAROCURE1173, IRGACURE784, IRGACURE OXE01, and IRGACURE OXE02 as a radical type polymerization initiator, IRGACURE250 and IRGACURE270 (all products are manufactured by BASF Co., Ltd.) as a cation type polymerization initiator, and the like are included.

Physical Property of Surface Protective Film

The crosslinked product (the surface protective film or the like according to this exemplary embodiment) which is obtained by performing crosslinking polymerization of the compound according to this exemplary embodiment is a material having excellent heat resistance as described above. Furthermore, the excellent heat resistance, specifically, means that excellent damage resistance is exhibited even under a high temperature environment.

Here, a heatproof temperature (a usable temperature range) of the crosslinked product which is obtained by performing crosslinking polymerization of the compound according to this exemplary embodiment is preferably from 60° C. to 200° C., and is more preferably from 80° C. to 160° C.

A water contact angle at 25° C. of the surface protective film according to this exemplary embodiment is preferably greater than or equal to 90°, and is more preferably greater than or equal to 100°.

Furthermore, the contact angle is measured by performing a θ/2 method with respect to a surface protective film sample applied on a film using water and a contact angle meter at 25° C. In addition, a contact angle with respect to hexadecane described later is measured by changing water to hexadecane.

A thickness of the surface protective film is not particularly limited, but is preferably from 1 μm to 500 μm, and is more preferably from 10 μm to 50 μm.

Use Application

In the surface protective film according to this exemplary embodiment thus obtained, the use application is not particularly limited insofar as it is assumed that a usage environment is at a temperature higher than a normal temperature (25° C.), and it is for an object in which a scratch may occur on a surface by being in contact with a foreign material.

For example, an endless belt or a roller for an image forming apparatus which is used for a fixing member, an intermediate transfer member, a recording medium feeding member, or the like of an image forming apparatus, a body or window glass of a vehicle, a photovoltaic solar cell panel or a panel reflecting solar light, and the like are included.

Endless Belt

Here, as an example of the use application of the surface protective film according to this exemplary embodiment, an endless belt for an image forming apparatus will be described.

The endless belt for an image forming apparatus according to this exemplary embodiment includes a belt-like base material, and the surface protective film according to this exemplary embodiment described above which is disposed on the belt-like base material.

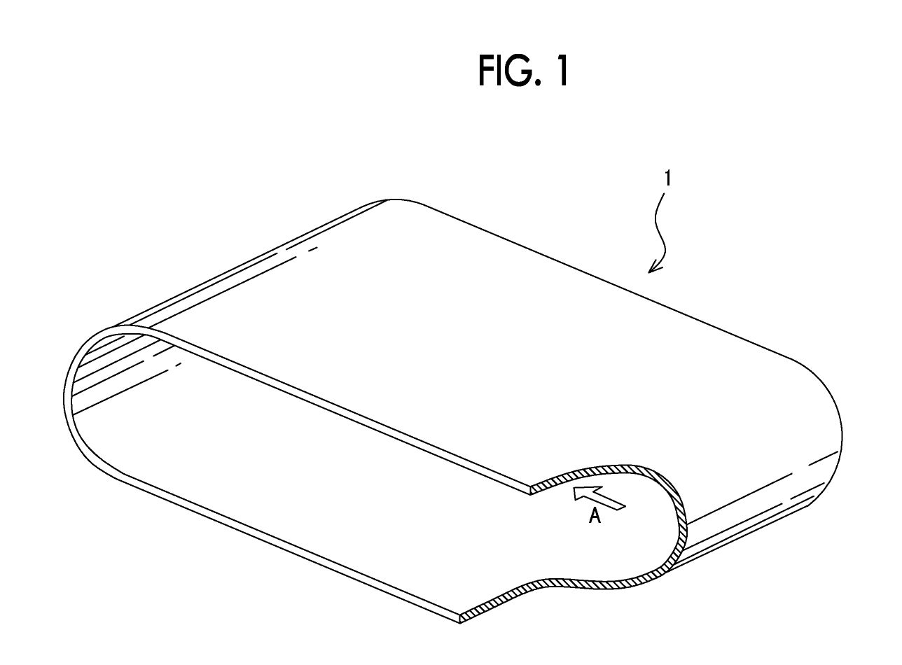

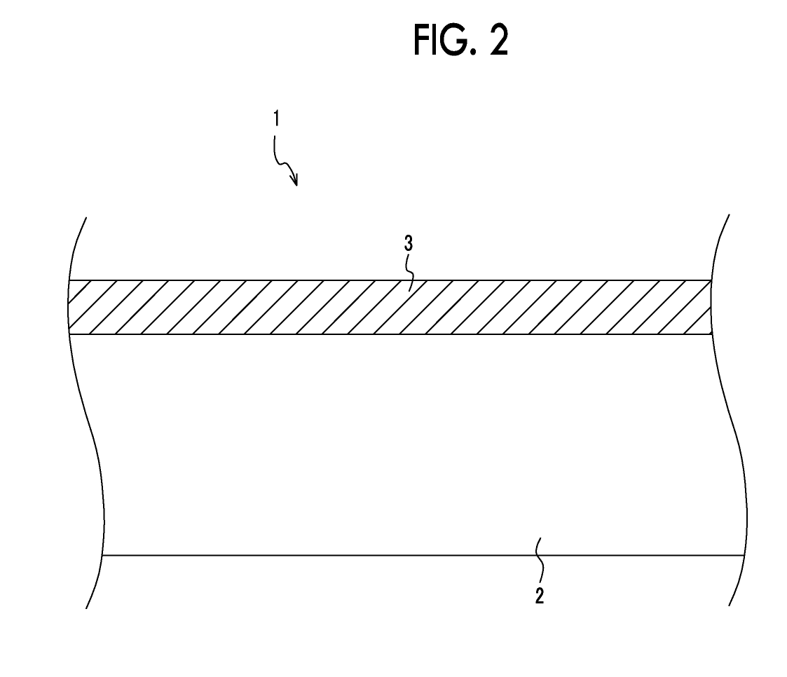

FIG. 1 is a perspective view (partially illustrated in a cross-sectional view) illustrating the endless belt according to this exemplary embodiment, and FIG. 2 is a cross-sectional view of the endless belt when viewed from a direction of an arrow A in FIG. 1.

As illustrated in FIG. 1 and FIG. 2, an endless belt 1 of this exemplary embodiment is an endless belt including a base material 2, and a surface layer 3 which is laminated on a surface of the base material 2.

Furthermore, as the surface layer 3, the surface protective film according to this exemplary embodiment described above is applied.

As a use application of the endless belt 1, for example, a fixing belt, an intermediate transfer belt, a recording medium feeding belt, and the like in the image forming apparatus are included.

Hereinafter, a case where the endless belt 1 is used as the fixing belt will be described.

As a material used for the base material 2, a material having heat resistance is preferable, and specifically, a material selected from various known plastic materials and metal materials is used.

Among the plastic materials, in general, a plastic material referred to as engineering plastic is preferable, and for example, a fluorine resin, a polyimide (PI), a polyamideimide (PAI), a polybenzimidazole (PBI), a polyether ether ketone (PEEK), a polysulfone (PSU), a polyethersulfone (PES), a polyphenylene sulfide (PPS), a polyetherimide (PEI), a wholly aromatic polyester (a liquid crystal polymer), and the like are preferable. In addition, among them, a thermosetting polyimide, a thermoplastic polyimide, a polyamideimide, a polyetherimide, a fluorine resin, and the like having excellent mechanical strength, heat resistance, abrasion resistance, chemical resistance, and the like are preferable.

In addition, the metal materials used for the base material 2 are not particularly limited, and as the metal materials, various metals or alloy materials are used, and for example, SUS, nickel, copper, aluminum, iron, and the like are preferably used. In addition, plural layers of heat resistant resins or metal materials may be laminated.

Hereinafter, a case where the endless belt 1 is used as the intermediate transfer belt or the recording medium feeding belt will be described.

As a material used for the base material 2, a polyimide resin, a polyamideimide resin, a polyester resin, a polyamide resin, a fluorine resin, and the like are included, and among them, a polyimide resin and a polyamideimide resin are preferably used. Furthermore, the base material may or may not include a joint insofar as it is in an annular shape (an endless shape), and a thickness of the base material 2 is preferably 0.02 mm to 0.2 mm in general.

When the endless belt 1 is used as the intermediate transfer belt or the recording medium feeding belt of the image forming apparatus, it is preferable that surface resistivity be controlled to be a range of 1×10⁹Ω/□ to 1×10¹⁴Ω/□ and volume resistivity be controlled to be a range of 1×10⁸ Ωcm to 1×10¹³ Ωcm. For this reason, as described above, carbon black such as Ketjen black, acetylene black, graphite, a metal or an alloy such as aluminum, nickel, and a copper alloy, metal oxide such as tin oxide, zinc oxide, potassium titanate, tin oxide-indium oxide or tin oxide-antimony oxide composite oxide, a conductive polymers such as a polyaniline, a polypyrrole, a polysulfone, and a polyacetylene, and the like are preferably added to the base material 2 or the surface layer 3 as a conductive agent (here, in the polymer, “conductivity” means that volume resistivity is less than 10⁷ Ω·cm). The conductive agent is independently used, or two or more conductive agents are used in combination.

Here, the surface resistivity and the volume resistivity are measured according to JIS-K6911 by using Hiresta UPMCP-450 type UR probe manufactured by TA Instruments under an environment of 22° C. and 55% RH.

When the endless belt 1 is used for fixing, the endless belt 1 may include an elastic layer between the base material 2 and the surface layer 3. As a material of the elastic layer, for example, various rubber materials are used. As the various rubber materials, for example, urethane rubber, ethylene-propylene rubber (EPM), silicone rubber, fluorine rubber (FKM), and the like are included, and in particular, silicone rubber having excellent heat resistance and workability is preferable. As the silicone rubber, for example, RTV silicone rubber, HTV silicone rubber, and the like are included, and specifically, polydimethyl silicone rubber (MQ), methyl vinyl silicone rubber (VMQ), methyl phenyl silicone rubber (PMQ), fluorosilicone rubber (FVMQ), and the like are included.

When the endless belt 1 is used as the fixing belt of an electromagnetic induction type fixing device, a heat-generating layer may be disposed between the base material 2 and the surface layer 3.

As a material used for the heat-generating layer, for example, nonmagnetic metal is included, and specifically, for example, a metal material such as gold, silver, copper, aluminum, zinc, tin, lead, bismuth, beryllium, antimony, and an alloy thereof (an alloy including these metals) is included.

A thickness of the heat-generating layer is preferably in a range of 5 μm to 20 μm, is more preferably in a range of 7 μm to 15 μm, and is especially preferably in a range of 8 μm to 12 μm.

Roller

The roller for an image forming apparatus according to this exemplary embodiment includes a cylindrical base material, and the surface protective film according to this exemplary embodiment described above which is disposed on the cylindrical base material.

Next, the roller according to this exemplary embodiment will be described. The roller of this exemplary embodiment is a cylindrical roller including a base material and a surface layer laminated on a surface of the base material.

Furthermore, as the surface layer, the surface protective film according to this exemplary embodiment described above is applied.

As a use application of the cylindrical roller, for example, a fixing roller, an intermediate transfer roller, a recording medium feeding roller, and the like in the image forming apparatus are included.

Hereinafter, a case where the cylindrical roller is used as the fixing roller will be described.

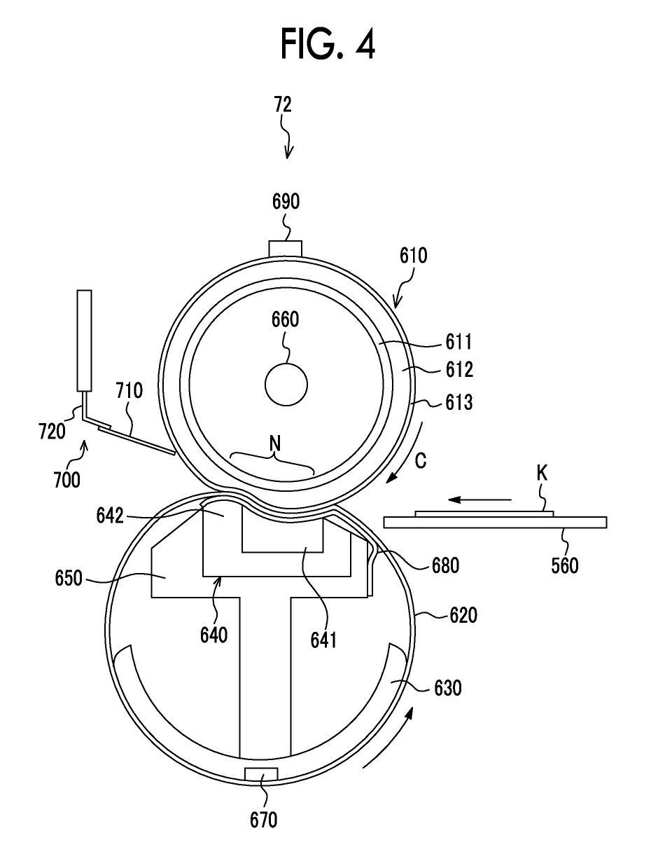

A shape, a structure, a size, and the like of a fixing roller 610 which is illustrated in FIG. 4 as the fixing member are not particularly limited, and the fixing roller 610 includes a surface layer 613 on a cylindrical core 611. In addition, as illustrated in FIG. 4, the fixing roller 610 may include an elastic layer 612 between the core 611 and the surface layer 613.

As a material of the cylindrical core 611, for example, metal such as aluminum (for example, an A-5052 material), SUS, iron, and copper, an alloy, a ceramic, FRM, and the like are included. In a fixing device 72 of this exemplary embodiment, the cylindrical core 611 is configured as a cylindrical member having an outer diameter of φ25 mm, a thickness of 0.5 mm, a length of 360 mm.

As a material of the elastic layer 612, a material selected from known materials is used, and any material may be used insofar as it is an elastic member having high heat resistance. In particular, as the material of the elastic layer 612, rubber having rubber hardness of approximately 15° to 45° (JIS-A), and an elastic member such as an elastomer are preferably used, and for example, silicone rubber, fluorine rubber and the like are included.

In this exemplary embodiment, among the materials, silicone rubber is preferable from a viewpoint of small surface tension and excellent elasticity. As the silicone rubber, for example, RTV silicone rubber, HTV silicone rubber, and the like are included, and specifically, polydimethyl silicone rubber (MQ), methyl vinyl silicone rubber (VMQ), methyl phenyl silicone rubber (PMQ), fluorosilicone rubber (FVMQ), and the like are included.

Furthermore, a thickness of the elastic layer 612 is preferably less than or equal to 3 mm, and is more preferably in a range of 0.5 mm to 1.5 mm. In the fixing device 72, the core is covered with HTV silicone rubber having rubber hardness of 35° (JIS-A) with a thickness of 72 μm.

A thickness of the surface layer 613, for example, is from 5 μm to 50 μm, and may be from 10 μm to 30 μm.

As a heating source heating the fixing roller 610, for example, a halogen lamp 660 is used, but the lamp is not particularly limited insofar as it has a shape or a structure which is contained in the core 611, and is selected according to a purpose. A surface temperature of the fixing roller 610 heated by the halogen lamp 660 is measured by a thermosensor 690 disposed in the fixing roller 610, and the temperature is controlled by a controller. The thermosensor 690 is not particularly limited, and for example, as the thermosensor 690, a thermistor, a temperature sensor, and the like are included.

Image Forming Apparatus

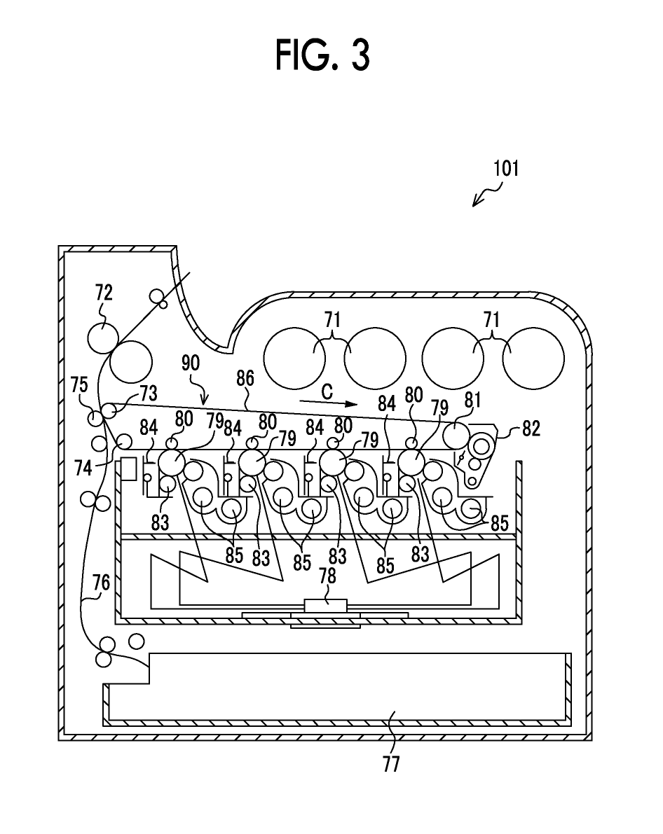

Next, the image forming apparatus of this exemplary embodiment using the endless belt of this exemplary embodiment and the roller of this exemplary embodiment will be described. FIG. 3 is a schematic view illustrating a main unit of a tandem type image forming apparatus including the endless belt according to this exemplary embodiment as a pressure belt of the fixing device, including the endless belt according to this exemplary embodiment as the intermediate transfer belt, and including the roller according to this exemplary embodiment as the fixing roller of the fixing device.

Specifically, an image forming apparatus 101 includes a photoreceptor 79 (an electrostatic latent image holding member), a charging roller 83 which charges a surface of the photoreceptor 79, a laser generator 78 (an electrostatic latent image forming unit) which exposes the surface of the photoreceptor 79 and forms an electrostatic latent image, a developing device 85 (a developing unit) which develops a latent image formed on the surface of the photoreceptor 79 by using a developer and forms a toner image, an intermediate transfer belt 86 (an intermediate transfer member) to which the toner image formed by the developing device 85 is transferred from the photoreceptor 79, a primary transfer roller 80 (a primary transfer unit) which transfers the toner image to the intermediate transfer belt 86, a photoreceptor cleaning member 84 which removes toner, dust, or the like attached to the photoreceptor 79, a secondary transfer roller 75 (a secondary transfer unit) which transfers the toner image on the intermediate transfer belt 86 to a recording medium, and the fixing device 72 (a fixing unit) which fixes the toner image on the recording medium. The photoreceptor 79 and the primary transfer roller 80 may be arranged immediately above the photoreceptor 79 as illustrated in FIG. 3, or may be arranged in a position which is separated from a position immediately above the photoreceptor 79.

Further, a configuration of the image forming apparatus 101 illustrated in FIG. 3 will be described in detail.

In the image forming apparatus 101, the charging roller 83, the developing device 85, the primary transfer roller 80 arranged via the intermediate transfer belt 86, and the photoreceptor cleaning member 84 are arranged around the photoreceptor 79 in a counterclockwise direction and a set of these members forma developing unit corresponding to one color. In addition, a toner cartridge 71 which replenishes the developing device 85 with the developer is disposed in each developing unit, and with respect to the photoreceptor 79 of each of the developing units, a laser generator 78 irradiating the surface of the photoreceptor 79 on an upstream side of the developing device 85, and a downstream side of the charging roller 83 (in a rotating direction of the photoreceptor 79) with laser light according to image information is disposed.

Four developing units corresponding to four colors (for example, cyan, magenta, yellow, and black) are arranged in series in a horizontal direction in the image forming apparatus 101, the intermediate transfer belt 86 is disposed to be inserted into a transfer region between the photoreceptor 79 and the primary transfer roller 80 of the four developing units. The intermediate transfer belt 86 is supported by a support roller 73, a support roller 74, and a driving roller 81 which are disposed on an inner surface side of the intermediate transfer belt 86 in the above order in the counterclockwise direction, and forms a belt supporting device 90. Furthermore, four primary transfer rollers are positioned on an upstream side of the support roller 74 and a downstream side of the support roller 73 (in a rotating direction of the intermediate transfer belt 86). In addition, a transfer member cleaning member 82 which cleans an outer circumferential surface of the intermediate transfer belt 86 is disposed on a side opposite to the driving roller 81 through the intermediate transfer belt 86 to be in contact with the driving roller 81.

In addition, the secondary transfer roller 75 for transferring the toner image formed on the outer circumferential surface of the intermediate transfer belt 86 onto a surface of recording paper transported from a sheet supply unit 77 through a sheet path 76 is disposed on a side opposite to the support roller 73 through the intermediate transfer belt 86 to be in contact with the support roller 73.

In addition, the sheet supply unit 77 containing a recording medium is disposed on a bottom portion of the image forming apparatus 101, and the recording medium is supplied to pass a contact portion between the support roller 73 and the secondary transfer roller 75 which configure the secondary transfer portion through the sheet path 76 from the sheet supply unit 77. The recording medium which has passed the contact portion is further transported by a feeding unit (not illustrated) to pass a contact portion of the fixing device 72, and is finally output to the outside of the image forming apparatus 101.

Next, an image forming method using the image forming apparatus 101 illustrated in FIG. 3 will be described. The toner image is formed in each of the developing units, and the surface of the photoreceptor 79 which is rotated in the counterclockwise direction is charged by the charging roller 83, then the latent image (the electrostatic latent image) is formed on the surface of the charged photoreceptor 79 by the laser generator 78 (an exposure device). Next, the latent image is developed by the developer supplied from the developing device 85, the toner image is formed, and the toner image which is transported to the contact portion between the primary transfer roller 80 and the photoreceptor 79 is transferred onto the outer circumferential surface of the intermediate transfer belt 86 which is rotated in a direction of an arrow C. Furthermore, toner, dust, or the like attached to the surface of the photoreceptor 79 after transferring the toner image is cleaned by the photoreceptor cleaning member 84, and the photoreceptor 79 prepares for forming the next toner image.

The toner image developed in each of the developing units having each color is transported to the secondary transfer portion in a state where the toner image is sequentially superimposed on the outer circumferential surface of the intermediate transfer belt 86 to correspond to the image information, and is transferred onto a recording paper surface which is transported from the sheet supply unit 77 through the sheet path 76 by the secondary transfer roller 75. The recording paper onto which the toner image is transferred is fixed by pressure heating when the recording paper further passes through the contact portion of the fixing device 72, and an image is formed on the recording medium surface, then the recording paper is output to the outside of the image forming apparatus.

Fixing Device (Image Fixing Device)

FIG. 4 is a schematic configuration diagram of the fixing device 72 which is disposed in the image forming apparatus 101 according to this exemplary embodiment. The fixing device 72 illustrated in FIG. 4 includes the fixing roller 610 as a rotating member which is rotary-driven, an endless belt 620 (a pressure belt), and a pressure pad 640 which is a pressure member pressurizing the fixing roller 610 through the endless belt 620. Furthermore, with the pressure pad 640, the endless belt 620 and the fixing roller 610 may be relatively pressured. Accordingly, the endless belt 620 side may be pressurized by the fixing roller 610, or the fixing roller 610 side may be pressurized by the endless belt 620.

Inside the fixing roller 610, the halogen lamp 660 as an example of a heating unit heating an unfixed toner image in a nipping region is disposed. The heating unit is not limited to the halogen lamp, and other heat-generating members generating heat may be used.

On the other hand, the thermosensor 690 is disposed in contact with a surface of the fixing roller 610. On the basis of a measured value of a temperature by the thermosensor 690, lighting of the halogen lamp 660 is controlled, and a surface temperature of the fixing roller 610 is maintained at a preset temperature (for example, 150° C.).

The endless belt 620 is rotatably supported by the pressure pad 640 and the belt travel guide 630 disposed inside the endless belt 620, and an edge guide (not illustrated). Then, the endless belt 620 is disposed in contact with the fixing roller 610 in a state where the endless belt 620 is pressurized with respect to the fixing roller 610 in a nipping region N.

The pressure pad 640 is disposed in a state where the pressure pad 640 is pressurized to the fixing roller 610 through the endless belt 620 inside the endless belt 620, and forms the nipping region N between the pressure pad 640 and the fixing roller 610. In the pressure pad 640, a pre-nipping member 641 for ensuring a wide nipping region N is disposed on an input port side of the nipping region N, and a peeling nipping member 642 for imparting strain to the fixing roller 610 is disposed on an output port side of the nipping region N.

Further, in order to decrease sliding resistance between an inner circumferential surface of the endless belt 620 and the pressure pad 640, a low friction sheet 680 is disposed on a surface in which the pre-nipping member 641 and the peeling nipping member 642 are in contact with the endless belt 620. Then, the pressure pad 640 and the low friction sheet 680 are retained by a metallic holder 650.

Further, a belt travel guide 630 is attached to the holder 650, and is configured such that the endless belt 620 is smoothly rotated. That is, the belt travel guide 630 scrapes against the inner circumferential surface of the endless belt 620, and thus the belt travel guide 630 is formed of a material having a small static friction coefficient. In addition, the belt travel guide 630 is formed of a material having low thermal conductivity such that it is difficult to take heat away from the endless belt 620.

Then, the fixing roller 610 is rotated in the direction of the arrow C by a driving motor (not illustrated), and according to the rotation, the endless belt 620 is rotated in a direction opposite to the rotating direction of the fixing roller 610. That is, while the fixing roller 610 is rotated in a clockwise direction in FIG. 4, the endless belt 620 is rotated in the counterclockwise direction.

A sheet K having the unfixed toner image thereon is guided by a fixing input guide 560, and is transported to the nipping region N. Then, when the sheet K passes through the nipping region N, the toner image on the sheet K is fixed by a pressure acting on the nipping region N, and heat supplied from the fixing roller 610.

In the fixing device 72, the nipping region N is ensured by the pre-nipping member 641 having a concave shape along an outer circumferential surface of the fixing roller 610.

In addition, by disposing the peeling nipping member 642 to protrude toward the outer circumferential surface of the fixing roller 610, the fixing device 72 according to this exemplary embodiment is configured such that strain of the fixing roller 610 locally increases in an output region of the nipping region N. According to this configuration, the sheet K after being fixed is peeled off from the fixing roller 610.

In addition, as an auxiliary unit of the peeling, a peeling member 700 is disposed on a downstream side of the nipping region N of the fixing roller 610. The peeling member 700 is retained by a holder 720 in a state where a peeling baffle 710 is in contact with the fixing roller 610 in a direction facing the rotating direction of the fixing roller 610 (a counter direction).

### Example

Hereinafter, the invention will be described with Examples in detail, but the invention is not limited to the following Examples. Furthermore, unless otherwise specifically noted, a “part” indicates a weight basis in the following description.

### Synthesis of Perfluoroalkylene Ether-Containing Compounds 1a and 1b

According to the following synthesis scheme, perfluoroalkylene ether-containing compounds 1a and 1b are synthesized. Furthermore, R^{X1} represented in the synthesis scheme of the perfluoroalkylene ether-containing compounds 1a and 1b indicates a hydrogen atom. In addition, a content of a hydroxyl group in a fluorine raw material 1 is 830 g/mol.

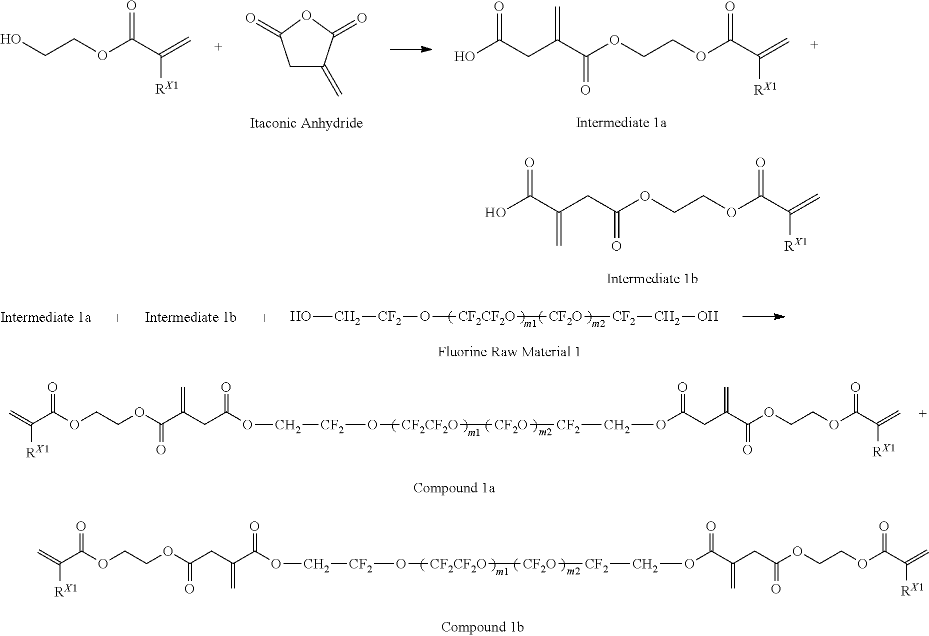

### Synthesis of Perfluoroalkylene Ether-Containing Compound 2

According to the following synthesis scheme, a perfluoroalkylene ether-containing compound 2 is synthesized. Furthermore, R^{X1} in the synthesis scheme of the perfluoroalkylene ether-containing compound 2 indicates a hydrogen atom. In addition, a content of a hydroxyl group in a fluorine raw material 2 is 830 g/mol.

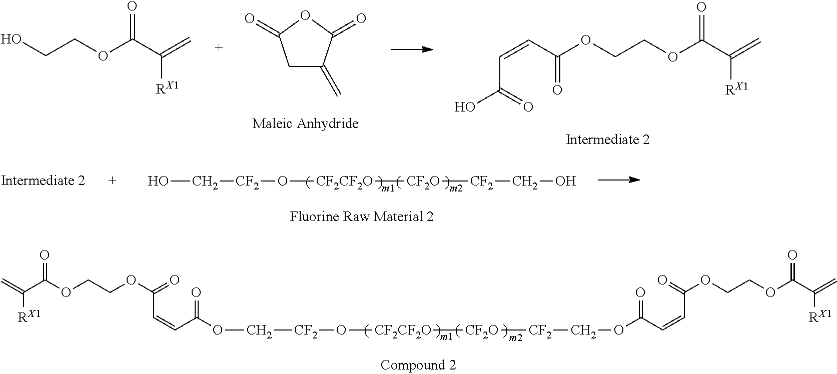

### Synthesis of Perfluoroalkylene Ether-Containing Compound 3

According to the following synthesis scheme, a perfluoroalkylene ether-containing compound 3 is synthesized. Furthermore, R^{X1} in the synthesis scheme of the perfluoroalkylene ether-containing compound 3 indicates a hydrogen atom. In addition, a content of a hydroxyl group in a fluorine raw material 3 is 830 g/mol.

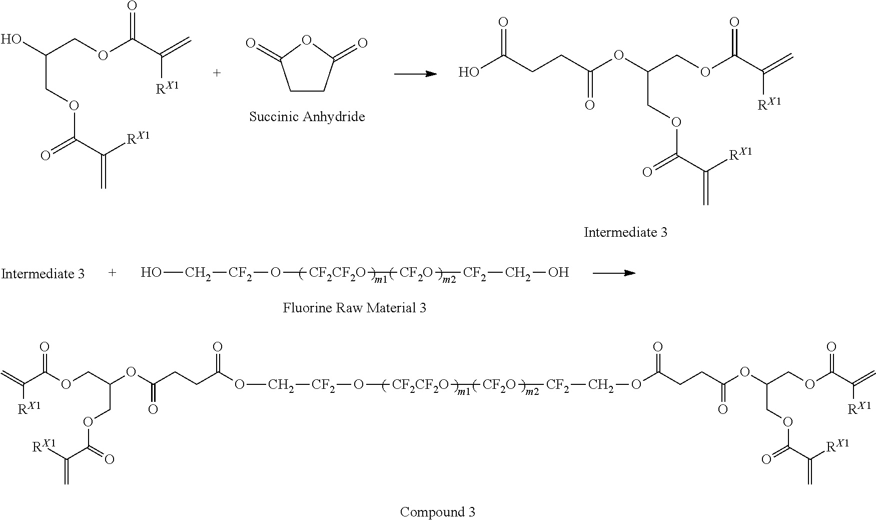

### Synthesis of Perfluoroalkylene Ether-Containing Compound 4

According to the following synthesis scheme, a perfluoroalkylene ether-containing compound 4 is synthesized. Furthermore, R^{X1} in the synthesis scheme of the perfluoroalkylene ether-containing compound 4 indicates a hydrogen atom. In addition, a content of a hydroxyl group in a fluorine raw material 4 is 830 g/mol.

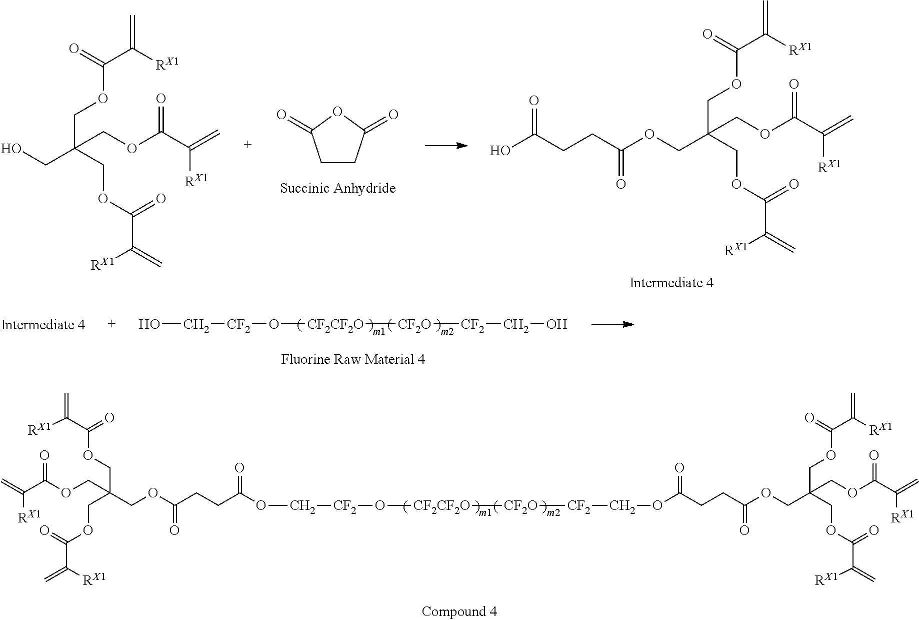

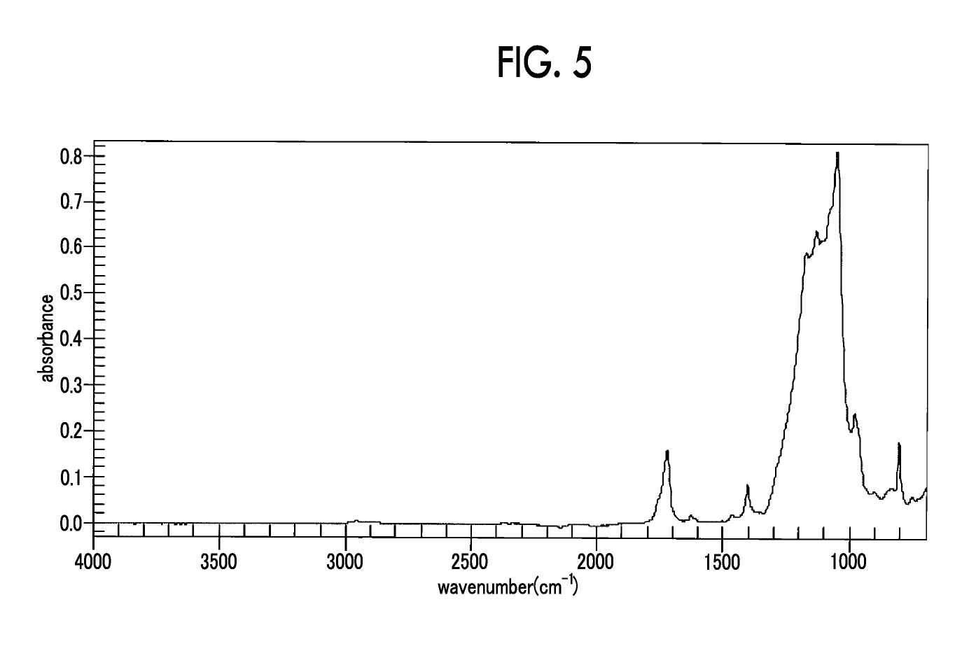

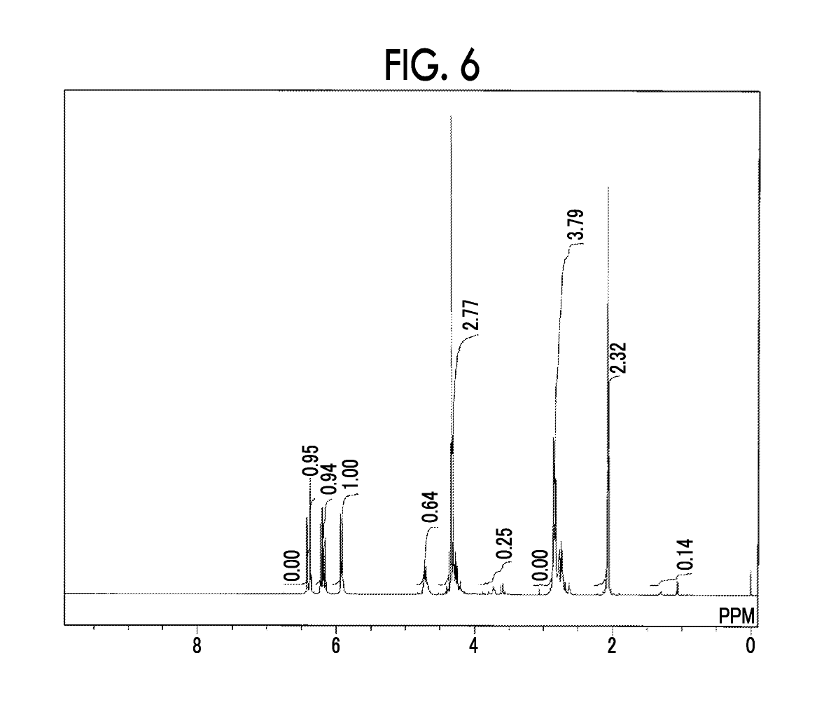

Here, as identification data of the perfluoroalkylene ether-containing compound 4 thus obtained, an IR chart is illustrated in FIG. 5, and a 1H-NMR chart is illustrated in FIG. 6.

### Synthesis of Perfluoroalkylene Ether-Containing Compound 5

According to the synthesis scheme of the compound 4, and the content of the hydroxyl group in the fluorine raw material 4 is changed to 1970 g/mol, and thus a perfluoroalkylene ether-containing compound 5 is synthesized. Furthermore, R^{X1} represented in the synthesis scheme indicates a hydrogen atom.

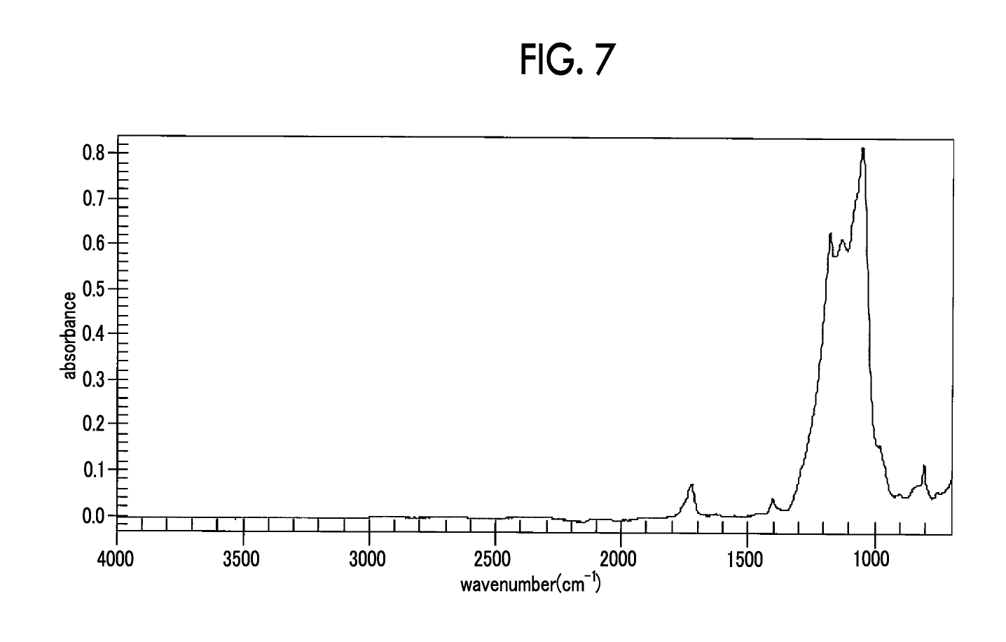

Here, as identification data of the perfluoroalkylene ether-containing compound 5 thus obtained, an IR chart is illustrated in FIG. 7.

### Synthesis of Perfluoroalkylene Ether-Containing Compound 6

According to the following synthesis scheme, a perfluoroalkylene ether-containing compound 6 is synthesized. Furthermore, R^{X1} in the synthesis scheme of the perfluoroalkylene ether-containing compound 6 indicates a hydrogen atom. In addition, a content of a hydroxyl group in a fluorine raw material 6 is 1970 g/mol.

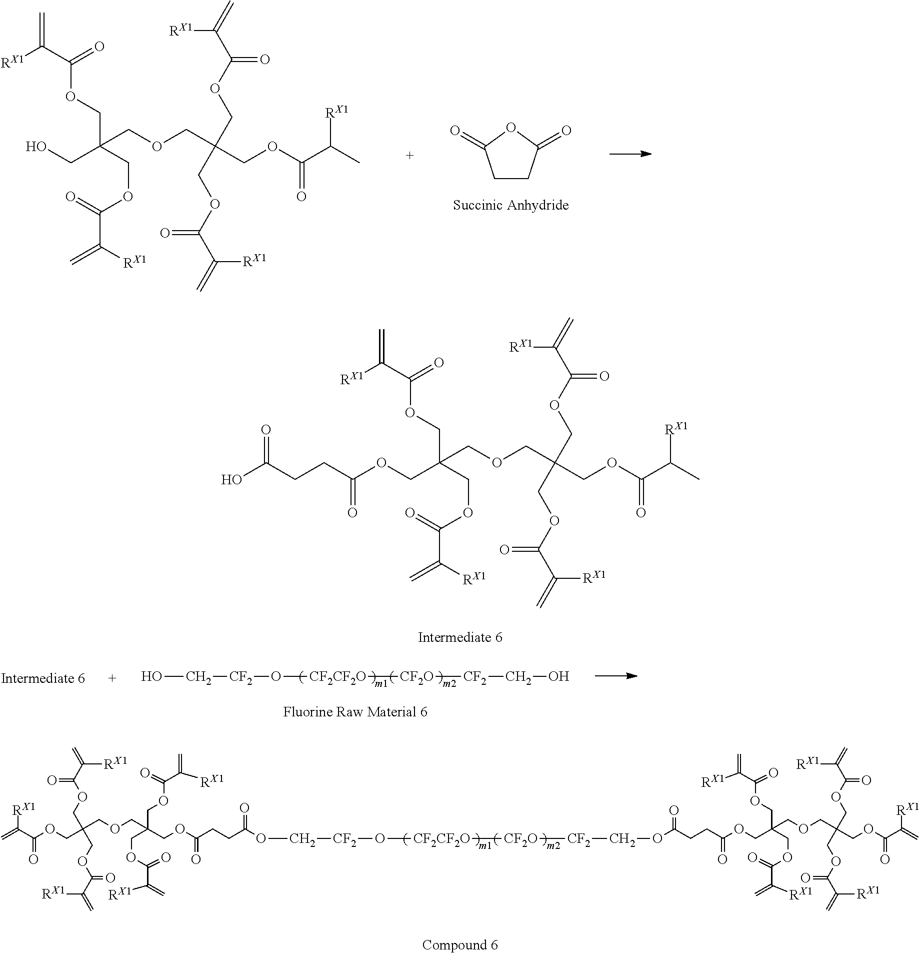

### Example 1

### Preparation of Coating Liquid for Forming Surface Protective Film

The following compositions are mixed, and a coating liquid is prepared.
Perfluoroalkylene ether-containing compounds 1a and 1b described above: 10 parts
Polymerization initiator (trade name: DAROCURE1173 manufactured by BASF Co., Ltd.): 0.1 parts
Solvent (2-butanone): 10 parts

Formation (Crosslinking Polymerization) of Surface Protective Film

The above-described coating liquid is applied (casted) onto a polyimide film having a thickness of 90 μm, is dried at 100° C. for 5 minutes, and thereby a solvent is volatilized, the coating liquid is irradiated with ultraviolet light by an ultraviolet curing device, and thus a hardened film is obtained. As an irradiation condition of the ultraviolet light, the ultraviolet light is emitted at light intensity of 1000 mmJ/cm² by using a high-pressure mercury vapor lamp under a nitrogen atmosphere (an oxygen concentration less than or equal to 1%).

### Example 2˜6

Surface protective films are formed by the method described in Example 1 except that the perfluoroalkylene ether-containing compounds 1a and 1b used in Example 1 is changed to each of the perfluoroalkylene ether-containing compounds 2, 3, 4, 5, and 6.

### Comparative Example 1

A surface protective film is formed by the method described in Example 1 except that the perfluoroalkylene ether-containing compound 1 used in Example 1 is changed to a compound having the following structure (a compound having a urethane bond and a perfluoroalkylene ether structure).

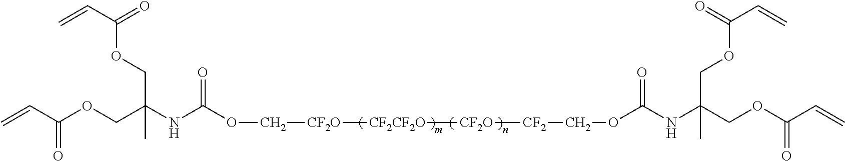

### Comparative Example 2

A surface protective film is formed by the method described in Example 1 except that the perfluoroalkylene ether-containing compound 1 used in Example 1 is changed to a compound having the following structure (a compound which has only one reactive crosslinking group in —B¹—X¹ and —B²—X² and does not have a group having a structure obtained by excluding X¹ or X² from the general formula (1)).

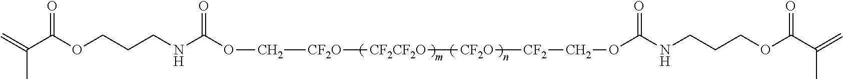

Evaluation

In order to evaluate damage resistance and heat resistance, surface protective film samples obtained in Examples and Comparative Examples described above are heated at 200° C. for 10 hours. The following evaluation is performed with respect to both samples, those before heating (initial) and those after heating.

Evaluation of Damage Resistance

The surface protective film samples obtained in Examples and Comparative Examples described above, and samples which are obtained by heating the surface protective film samples at 200° C. for 10 hours are subjected to a scratching test by using a scratching hardness meter (manufactured by ERICHSEN Co., Ltd., a tip diameter of 0.75 mm) at a normal temperature (25° C.) under a load of 2 N, and are heated at 80° C. for 30 seconds, then a scratched portion is observed, and thus presence or absence of a flaw is evaluated.

B: Flaw in protective film sample

A: No flaw in protective film sample

Evaluation of Contact Angle

Contact angles of the surface protective film samples obtained in Examples and Comparative Examples described above, and samples which are obtained by heating the surface protective film samples at 200° C. for 10 hours are measured by water or hexadecane. Furthermore, the measurement of the contact angle described above is performed by a θ/2 method at 25° C. using a contact angle meter (model number: CA-S-Rugata manufactured by Kyowa Interface Science Co., LTD.). Results are shown in Table 1.

Toner Peeling Property

The surface protective film samples obtained in Examples and Comparative Examples described above, and samples which are obtained by heating the surface protective film samples at 200° C. for 10 hours are attached to a surface of a fixing roller of a fixing machine, a sheet of paper on which an unfixed black solid image is formed is fed, and fixibility is confirmed. Furthermore, as the fixing machine a fixing machine manufactured by Fuji Xerox Co., Ltd., trade name: DocuCentre C2101 is used. An evaluation criterion is as follows, and results are shown in Table 1.

C: Toner attached to entire surface of protective film sample

B: Toner attached to approximately half of protective film sample

A: No toner attached to protective film sample

IR Evaluation (Difference in IR Chart Between Initial and After Heating)

Spectrums of the surface protective film samples obtained in Examples and Comparative Examples described above, and samples which are obtained by heating the surface protective film samples at 200° C. for 10 hours are measured by using ATR-IR, differences in IR charts between before heating (initial) and after heating are compared. Furthermore, as an ATR prism, a diamond prism is used.

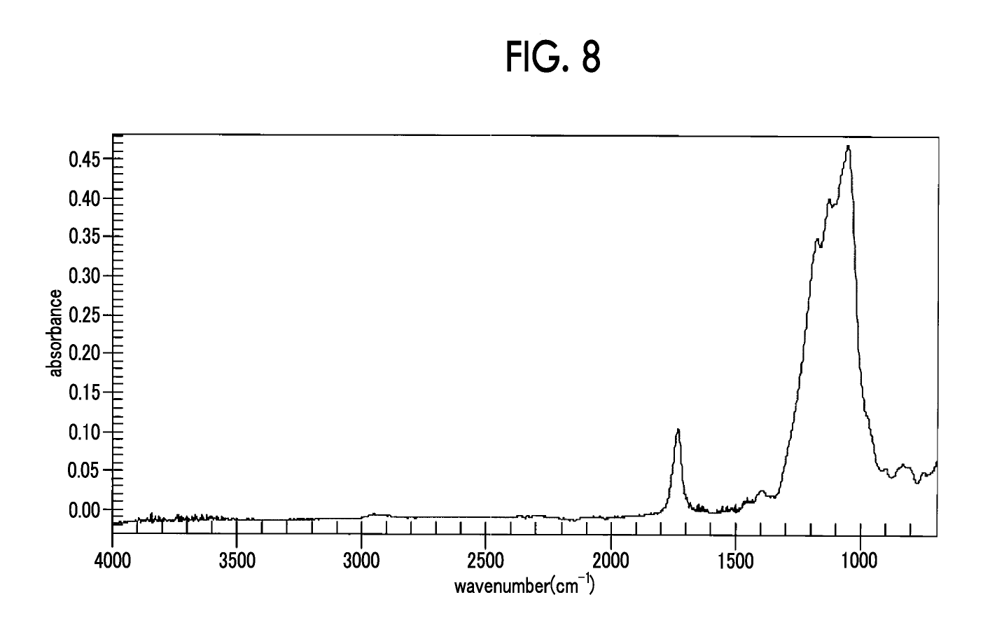

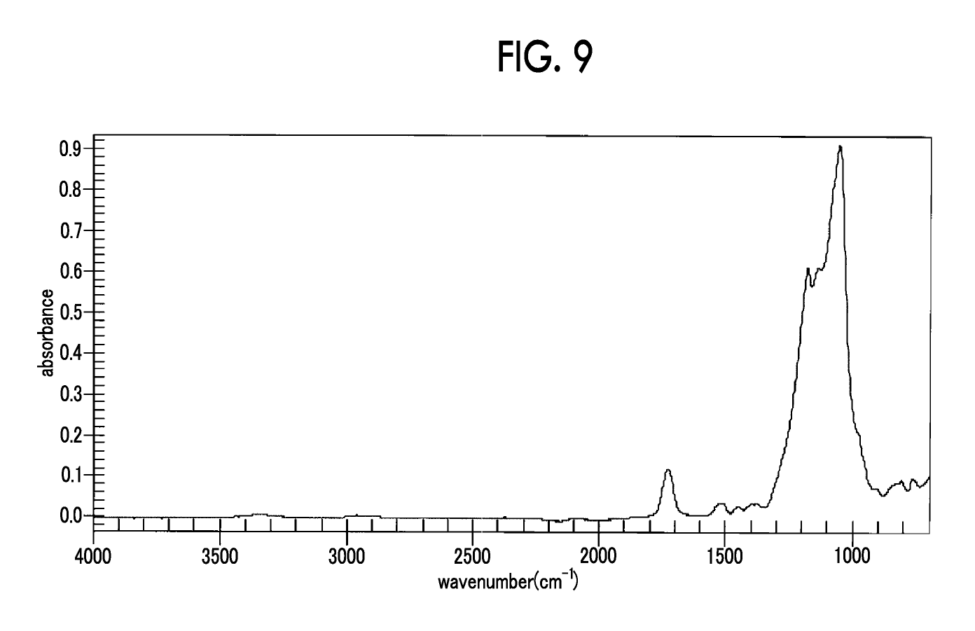

The IR chart of the surface protective film samples (before heating (initial)) obtained in Example 4 and Comparative Example 1 is illustrated in FIG. 8 and FIG. 9.

TABLE 1

| | | Evaluation | | | | | | | | |
|---|---|---|---|---|---|---|---|---|---|---|
| | | Initial | | | | After Heating | | | | Difference in IR Chart |
| | | Damage | Contact Angle (°) | | Toner Peeling | Damage | Contact Angle (°) | | Toner Peeling | Between Initial and After |
| | | Resistance | Water | Hexadecane | Property | Resistance | Water | Hexadecane | Property | Heating |
| Example | 1 | A | 109 | 60 | A | A | 109 | 57 | A | No Difference in IR |
| | | | | | | | | | | Chart |
| | 2 | A | 109 | 60 | A | A | 109 | 57 | A | No Difference in IR |
| | | | | | | | | | | Chart |
| | 3 | A | 110 | 66 | A | A | 110 | 65 | A | No Difference in IR |
| | | | | | | | | | | Chart |
| | 4 | A | 110 | 64 | A | A | 110 | 64 | A | No Difference in IR |
| | | | | | | | | | | Chart |
| | 5 | A | 110 | 62 | A | A | 110 | 62 | A | No Difference in IR |
| | | | | | | | | | | Chart |
| | 6 | A | 110 | 64 | A | A | 110 | 64 | A | No Difference in IR |
| | | | | | | | | | | Chart |
| Comparative | 1 | A | 110 | 62 | A | B | 105 | 51 | C | Urethane-Derived Peak |
| Example | | | | | | | | | | Dissipation After Heating |
| | 2 | B | 108 | 60 | B | B | 101 | 50 | C | Urethane-Derived Peak |
| | | | | | | | | | | Dissipation After Heating |

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A perfluoroalkylene ether-containing compound represented by the following general formula (1): 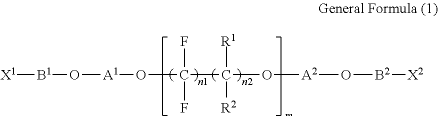
wherein in the general formula (1), R¹ and R² each independently indicates a fluorine atom or a trifluoromethyl group, provided that both R¹ and R² are not the fluorine atom;
n1 indicates an integer from 1 to 5, n2 indicates an integer from 0 to 2, and the total number of n1 and n2 is less than or equal to 5; m indicates an integer greater than or equal to 1;
A¹ and A² each independently indicates a bivalent group represented by the following general formula (2);
B¹ and B² each independently indicates a bivalent group selected from the group consisting of a single bond and the following (B-1) to (B-3);
X¹ and X² each independently indicates a monovalent group having at least one reactive crosslinking group selected from the group consisting of the following (X-1) to (X-8); X¹ and X² may each independently have one or more groups having a structure obtained by excluding X¹ or X² from the general formula (1); provided that when B¹ is a single bond or the following (B-1), X¹ indicates a monovalent group having two or more reactive crosslinking groups or one or more reactive crosslinking groups and one or more groups having a structure obtained by excluding X¹ from the general formula (1), and when B² is a single bond or the following (B-1), X² indicates a monovalent group having two or more reactive crosslinking group or one or more reactive crosslinking groups and one or more groups having a structure obtained by excluding X² from the general formula (1), 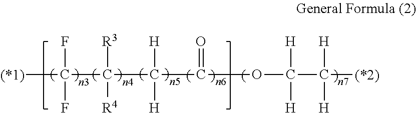
wherein in the general formula (2), R³ and R⁴ each independently indicates a fluorine atom or a trifluoromethyl group, provided that both R³ and R⁴ are not the fluorine atom;
n3 indicates an integer from 0 to 5, n4 indicates an integer from 0 to 2, n5 indicates an integer greater than or equal to 0, n6 indicates 0 or 1, and n7 indicates an integer greater than or equal to 0, provided that all of n3, n4, n5, and n6 are not 0;
a bivalent group represented by the general formula (2) is bonded to a perfluoroalkylene ether structure in a (*1) portion; 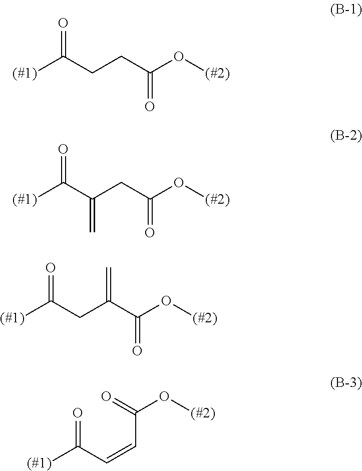
(B-1) to (B-3) are respectively bonded to X¹ or X² in a (#2) portion; 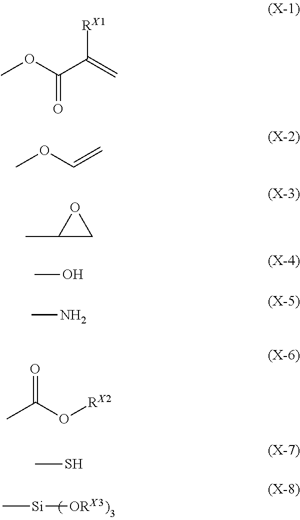
R^{X1} in (X-1) indicates a hydrogen atom, a methyl group, or a trifluoromethyl group; R^{X2} in (X-6) indicates a hydrogen atom or an alkyl group; and R^{X3} in (X-8) indicates an alkyl group.

2. The perfluoroalkylene ether-containing compound according to claim 1, wherein in the general formula (1), n1 is an integer from 1 to 3, n2 is 0 or 1, and the total number of n1 and n2 is from 1 to 3.

3. The perfluoroalkylene ether-containing compound according to claim 1, wherein in the general formula (1), m is an integer from 2 to 100.

4. The perfluoroalkylene ether-containing compound according to claim 1, wherein in the general formula (1), m is an integer from 5 to 50.

5. The perfluoroalkylene ether-containing compound according to claim 1, wherein in the general formula (2), the total number of n5 and n6 is less than or equal to 2.

6. The perfluoroalkylene ether-containing compound according to claim 1, wherein in the general formula (2), the total number of n5 and n6 is less than or equal to 1.

7. The perfluoroalkylene ether-containing compound according to claim 1, wherein the bivalent group represented by A¹ and A² is selected from (A-1) to (A-12): 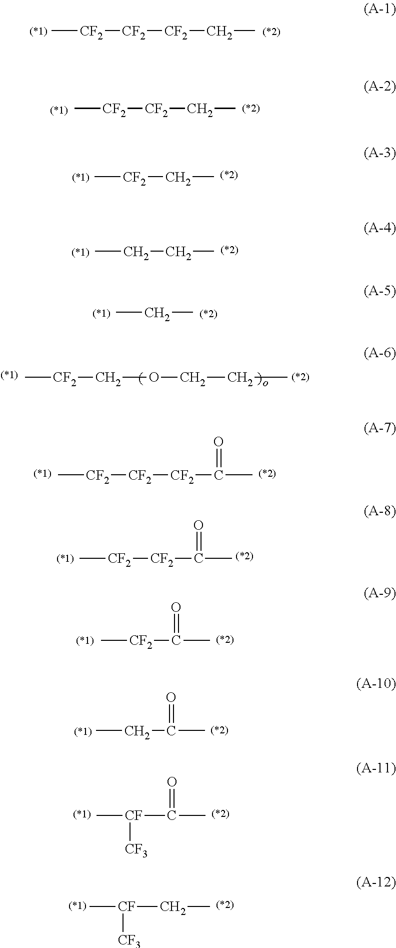
wherein the bivalent group is bonded to a perfluoroalkylene ether structure of the general formula (1) in a (*1) portion.

8. The perfluoroalkylene ether-containing compound according to claim 1, wherein in the general formula (1), B¹ and B² each is a bivalent group represented by (B-1).

9. A surface protective film having a crosslinked polymer structure of the perfluoroalkylene ether-containing compound according to claim 1.
